(19) United States
(12) Patent Application Publication
(10) Pub. No.: US 2025/0008759 A1
(43) Pub. Date: Jan. 2, 2025

(54) DETECTION DEVICE

(71) Applicant: Japan Display Inc., Tokyo (JP)

(72) Inventors: Makoto HAYASHI, Tokyo (JP); Hirofumi KATO, Tokyo (JP); Yoshitaka KOJIMA, Tokyo (JP); Yuta HAGA, Tokyo (JP); Ayato KITAMURA, Tokyo (JP); Yoshiyuki TERADA, Tokyo (JP)

(21) Appl. No.: 18/883,461

(22) Filed: Sep. 12, 2024

Related U.S. Application Data
(63) Continuation of application No. PCT/JP2023/009545, filed on Mar. 13, 2023.

(30) Foreign Application Priority Data
Mar. 24, 2022 (JP) 2022-049126

(51) Int. Cl. H10K 39/00; A61B 5/024; A61B 5/1455

(52) U.S. Cl. CPC H10K 39/621; A61B 5/02416; A61B 5/14551

(57) ABSTRACT

According to an aspect, a detection device includes: a light source configured to emit light to an object to be detected; a plurality of photodiodes that each include a sensor electrode and an organic semiconductor layer and are arranged in a detection area; and one or a plurality of detection circuits coupled to the photodiodes. The photodiodes includes a first photodiode and a second photodiode that has a shorter distance from the light source than that of the first photodiode. A light-receiving area of the first photodiode is larger than a light-receiving area of the second photodiode.

### CROSS-REFERENCE TO RELATED APPLICATION

This application claims the benefit of priority from Japanese Patent Application No. 2022-049126 filed on Mar. 24, 2022 and International Patent Application No. PCT/JP2023/009545 filed on Mar. 13, 2023, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (refer to, for example, Japanese Patent Application Laid-open Publication No. 2009-032005). Among such optical sensors, sensors are known each including a plurality of photodiodes in which an organic semiconductor material is used as an active layer. Light emitted from a light source is scattered and reflected by an object to be detected and enters each of the photodiodes. Thus, the photodiodes detect biometric information on the object to be detected.

The amount of light entering the photodiodes varies depending on the difference in distance between the light source and the photodiodes; for example, a photodiode disposed farther from the light source may not receive a sufficient amount of light as compared with a photodiode disposed closer to the light source.

For the foregoing reasons, there is a need for a detection device capable of improving accuracy of detection.

### SUMMARY

According to an aspect, a detection device includes: a light source configured to emit light to an object to be detected; a plurality of photodiodes that each include a sensor electrode and an organic semiconductor layer and are arranged in a detection area; and one or a plurality of detection circuits coupled to the photodiodes. The photodiodes includes a first photodiode and a second photodiode that has a shorter distance from the light source than that of the first photodiode. A light-receiving area of the first photodiode is larger than a light-receiving area of the second photodiode.

### BRIEF DESCRIPTION OF THE DRAWINGS

FIG. 1 is a plan view illustrating a detection device according to a first embodiment;

FIG. 2 is a sectional view illustrating an exemplary use of the detection device according to the first embodiment for measuring biometric information;

FIG. 3 is a sectional view along III-III′ of FIG. 1;

FIG. 4 is a plan view illustrating a detection device according to a first modification of the first embodiment;

FIG. 5 is a plan view illustrating a detection device according to a second modification of the first embodiment;

FIG. 6 is a plan view illustrating a detection device according to a third modification of the first embodiment;

FIG. 7 is a plan view illustrating a detection device according to a second embodiment;

FIG. 8 is a plan view illustrating a detection device according to a fourth modification of the second embodiment;

FIG. 9 is a plan view illustrating a detection device according to a third embodiment;

FIG. 10 is a plan view illustrating a detection device according to a fifth modification of the third embodiment;

FIG. 11 is a sectional view illustrating an exemplary use of a detection device according to a fourth embodiment for measuring the biometric information;

FIG. 12 is a plan view illustrating the detection device according to the fourth embodiment;

FIG. 13 is a plan view illustrating a detection device according to a sixth modification of the fourth embodiment;

FIG. 14 is a plan view illustrating a detection device according to a seventh modification of the fourth embodiment;

FIG. 15 is a plan view illustrating a detection device according to a fifth embodiment;

FIG. 16 is a block diagram illustrating a configuration example of the detection device according to the fifth embodiment;

FIG. 17 is a circuit diagram illustrating the detection device according to the fifth embodiment;

FIG. 18 is a circuit diagram illustrating a partial detection area of the detection device according to the fifth embodiment;

FIG. 19 is a plan view illustrating an arrangement relation between a plurality of photodiodes and a plurality of light sources in the detection device according to the fifth embodiment;

FIG. 20 is a plan view illustrating an arrangement relation between the photodiodes and the light sources in a detection device according to an eighth modification of the fifth embodiment; and

FIG. 21 is a plan view illustrating an arrangement relation between the photodiodes and the light sources in a detection device according to a ninth modification of the fifth embodiment.

### DETAILED DESCRIPTION

The following describes modes (embodiments) for carrying out the present disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### First Embodiment

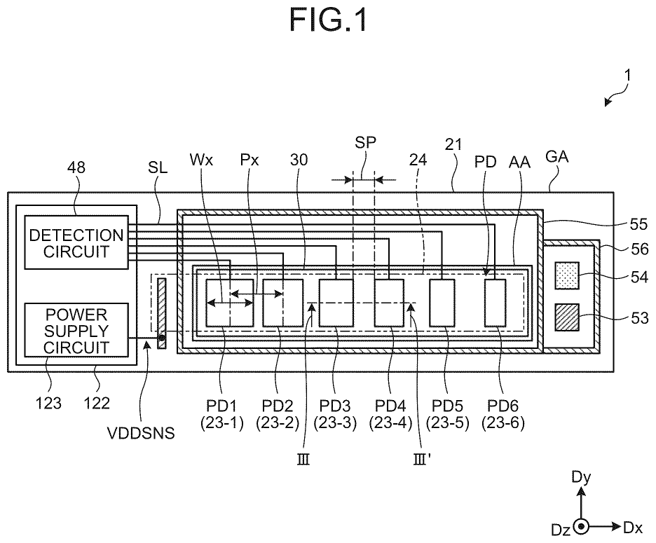

FIG. 1 is a plan view illustrating a detection device according to a first embodiment. As illustrated in FIG. 1, a detection device 1 includes a substrate 21, a plurality of photodiodes PD, a plurality of signal lines SL, a plurality of light sources 53 and 54, light-blocking walls 55 and 56, and a control circuit 122.

The substrate 21 has a detection area AA and a peripheral area GA. The detection area AA is an area provided with the photodiodes PD. The peripheral area GA is an area between the outer perimeter of the detection area AA and the ends of the substrate 21, and is an area not provided with the photodiodes PD. The signal lines SL, the light sources 53 and 54, and the control circuit 122 are provided in the peripheral area GA of the substrate 21.

In the following description, a first direction Dx is one direction in a plane parallel to the substrate 21. A second direction Dy is one direction in the plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the substrate 21. The term “plan view” refers to a positional relation when viewed in a direction orthogonal to the substrate 21.

The detection device 1 includes the photodiodes PD as optical sensor elements. Each of the photodiodes PD outputs an electrical signal corresponding to light received by the photodiode PD. More specifically, the photodiode PD is an organic photodiode (OPD) using an organic semiconductor. The photodiodes PD are arranged in the first direction Dx in the detection area AA.

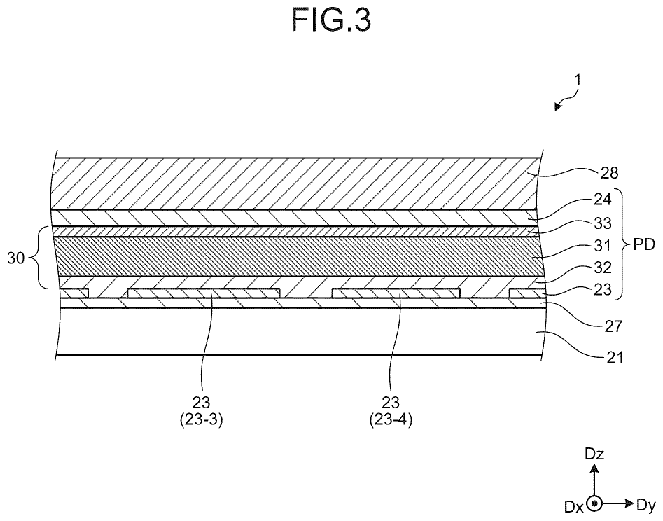

The photodiodes PD include an organic semiconductor layer 30 (a lower buffer layer 32, an active layer 31, and an upper buffer layer 33 (refer to FIG. 3)), lower electrodes 23 arranged below the organic semiconductor layer 30, and an upper electrode 24 disposed on the organic semiconductor layer 30. The photodiodes PD are arranged in the first direction Dx in the detection area AA, and a plurality of the lower electrodes 23 are respectively provided for the photodiodes PD. The lower electrodes 23 are arranged apart from one another in the first direction Dx. The upper electrode 24 is provided extending across the photodiodes PD and provided continuously in the detection area AA. The configuration of the photodiodes PD, the lower electrodes 23, and the upper electrode 24 will be described later with reference to FIG. 3.

The signal lines SL are electrically coupled to the respective lower electrodes 23 of the photodiodes PD. Each of the signal lines SL extends in the second direction Dy from a coupling point with a corresponding one of the lower electrodes 23, bends in the first direction Dx, and extends in the first direction Dx along the arrangement direction of the photodiodes PD. The portions of the signal lines SL extending in the first direction Dx are arranged in the second direction Dy. Each of the signal lines SL is coupled to a detection circuit 48 included in the control circuit 122. In other words, the detection circuit 48 is electrically coupled to the lower electrodes 23 of the photodiodes PD through the signal lines SL.

The light sources 53 and 54 are arranged adjacent to the photodiodes PD in the first direction Dx in the peripheral area GA outside the detection area AA. In the present embodiment, the photodiodes PD are arranged between the control circuit 122 and the light sources 53 and 54 in the first direction Dx. The light source 53 and the light source 54 are arranged in the second direction Dy. For example, inorganic light-emitting diodes (LEDs) or organic electroluminescent (EL) diodes (organic light-emitting diodes (OLEDs)) are used as the light sources 53 and 54.

The light source 53 and the light source 54 emit light having different wavelengths from each other. For example, the light source 53 emits red light and the light source 54 emits infrared light. However, the light emitted by the light source 53 and the light source 54 are not limited thereto and may be light having other wavelengths, such as blue and green light. The wavelengths of the light emitted from the light source 53 and the light source 54 are appropriately selected according to the type of an object to be detected Fg and the type of biometric information to be detected.

The arrangement of the light sources 53 and 54 illustrated in FIG. 1 is merely an example and can be changed as appropriate. The detection device 1 is provided with a plurality of types of the light sources 53 and 54 as light sources. However, the light sources are not limited thereto and may be of one type. The light sources 53 and 54 are provided on the same substrate 21 as the photodiodes PD but may be provided on a substrate different from the substrate 21. Moreover, the number of the light sources 53 and 54 is not limited to being larger than one, and at least one light source only needs to be disposed.

The light-blocking wall 55 is provided so as to surround the photodiodes PD in plan view. The light-blocking wall 56 is provided so as to surround the light sources 53 and 54. The light-blocking walls 55 and 56 can reduce the entrance of extraneous light different from the light emitted from the light sources 53 and 54 onto the photodiodes PD.

The control circuit 122 (the detection circuit 48 and a power supply circuit 123) is disposed adjacent to the photodiodes PD in the first direction Dx in the peripheral area GA of the substrate 21. The control circuit 122 is a circuit that supplies control signals to the photodiodes PD to control detection operations. Each of the photodiodes PD outputs, as a detection signal Vdet, the electrical signal corresponding to the light received by the photodiodes PD to the detection circuit 48. In the present embodiment, the detection signals Vdet of the photodiodes PD are sequentially output to the detection circuit 48 in a time-divisional manner. Thereby, the detection device 1 detects information on the object to be detected based on the detection signals Vdet from the photodiodes PD.

The power supply circuit 123 supplies a sensor power supply signal (sensor power supply voltage) VDDSNS to the upper electrode 24 of the photodiode PD. The power supply circuit 123 supplies a power supply voltage to the light sources 53 and 54.

The control circuit 122 (detection circuit 48 and power supply circuit 123) is provided in the peripheral area GA opposite the light sources 53 and 54. However, the arrangement of the control circuit 122 (detection circuit 48 and power supply circuit 123) is not limited thereto and can be changed as appropriate. The control circuit 122 (detection circuit 48 and power supply circuit 123) is provided on the same substrate 21 as the photodiodes PD, but is not limited to this configuration. The control circuit 122 (detection circuit 48 and power supply circuit 123) may be provided on another control substrate coupled to the substrate 21, for example, through a flexible printed circuit board or the like. The detection circuit 48 and the power supply circuit 123 may each be formed as an individual circuit.

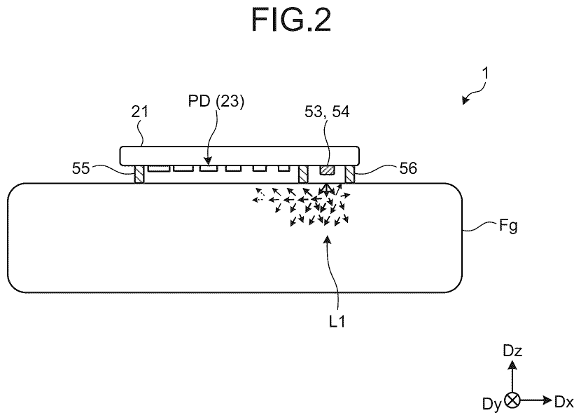

FIG. 2 is a sectional view illustrating an exemplary use of the detection device according to the first embodiment for measuring the biometric information. As illustrated in FIG. 2, the detection device 1 is used, for example, to measure the object to be detected Fg, such as a finger or a wrist. A surface of the substrate 21 on which the photodiodes PD and the light sources 53 and 54 are provided, is disposed so as to face the object to be detected Fg. The light-blocking walls 55 and 56 are provided in contact with the object to be detected Fg, whereby the photodiodes PD and the light sources 53, 54 are arranged in an area surrounded by the substrate 21, the object to be detected Fg, and the light-blocking walls 55 and 56. The light-blocking wall 55 blocks light between the photodiodes PD and the light sources 53, 54.

Light L1 from the light sources 53 and 54 is emitted toward the inside of the object to be detected Fg, scattered and reflected in the object to be detected Fg, and enters the photodiodes PD. Thereby, the detection device 1 can detect, based on the light L1, pulse waves and a vascular image (vascular pattern) as information on a living body. Alternatively, the detection device 1 can detect a blood oxygen saturation level, in addition to the pulse waves, pulsation, and the vascular image, as the information on a living body. The detection device 1 may be formed, for example, in a ring shape annularly provided around a finger (refer to FIG. 11). Alternatively, the detection device 1 can be employed in a smartwatch or a wearable device, for example. The arrangement of the photodiodes PD and the light sources 53 and 54 illustrated in FIGS. 1 and 2 is merely exemplary, and can be changed as appropriate depending on an apparatus on which the detection device 1 is mounted.

As illustrated in FIGS. 1 and 2, the photodiodes PD are arranged at different distances from the light sources 53 and 54. That is, the amount of the light L1 incident on the photodiode PD closer to the light sources 53 and 54 is larger, and the amount of the light L1 incident on the photodiode PD further away from the light sources 53 and 54 is smaller. Although FIGS. 1 and 2 illustrate six photodiodes PD for ease of explanation, the variation of the amount of the light L1 incident on the photodiodes PD increases as the number of the photodiodes PD increases, that is, as the sensor area (area of the detection area AA) increases.

As illustrated in FIG. 1, a plurality of photodiodes PD1, PD2, PD3, PD4, PD5, and PD6 are arranged in this order in the first direction Dx. Lower electrodes 23-1, 23-2, 23-3, 23-4, 23-5, and 23-6 are provided for the photodiodes PD1, PD2, PD3, PD4, PD5, and PD6, respectively. The photodiode PD1 is disposed farthest from the light sources 53 and 54, and the distance from the light sources 53 and 54 decreases in the order of the photodiodes PD1, PD2, PD3, PD4, PD5, and PD6.

In the following description, the photodiodes PD1, PD2, PD3, PD4, PD5, and PD6 are each simply referred to as the photodiode PD when they need not be distinguished from one another. The lower electrodes 23-1, 23-2, 23-3, 23-4, 23-5, and 23-6 are each simply referred to as the lower electrode 23 when they need not be distinguished from one another.

In the present embodiment, the photodiodes PD are formed such that: the photodiode PD, which has a longer distance from the light sources 53 and 54, has a larger light-receiving area; and the photodiode PD, which has a shorter distance from the light sources 53 and 54, has a smaller light-receiving area. Specifically, the light-receiving area of the photodiode PD1 (first photodiode) is larger than that of the photodiode PD2 (second photodiode) having a shorter distance from the light sources 53 and 54 than that of the photodiode PD1. The light-receiving area of the photodiode PD2 is larger than that of the photodiode PD3 (third photodiode) having a shorter distance from the light sources 53 and 54 than that of the photodiode PD2. Similarly, the light-receiving area decreases in the order of the photodiodes PD1, PD2, PD3, PD4, PD5, and PD6.

The light-receiving area of the photodiode PD corresponds to the area of a region that substantially serves as an optical sensor when the photodiode PD is irradiated with the light L1. That is, the light-receiving area of the photodiode PD corresponds to the area of a portion where the lower electrode 23, the organic semiconductor layer 30, and the upper electrode 24 forming the photodiode PD overlap one another. In other words, a region where only one of the lower electrode 23 and the organic semiconductor layer 30 is stacked, is not included in the light-receiving area of the photodiode PD.

In the present embodiment, as illustrated in FIG. 1, the area of the lower electrode 23 decreases in the order of the lower electrodes 23-1, 23-2, 23-3, 23-4, 23-5, and 23-6. That is, the area of the lower electrode 23-1 (sensor electrode) of the photodiode PD1 (first photodiode) is larger than that of the lower electrode 23-2 (sensor electrode) of the photodiode PD2 (second photodiode) having a shorter distance from the light sources 53 and 54 than that of the photodiode PD1. The area of the lower electrode 23-2 of the photodiode PD2 is larger than that of the lower electrode 23-3 (sensor electrode) of the photodiode PD3 (third photodiode) having a shorter distance from the light sources 53 and 54 than that of the photodiode PD2. Similarly, the electrode area decreases in the order of the lower electrodes 23-1, 23-2, 23-3, 23-4, 23-5, and 23-6.

In more detail, a width Wx in the first direction Dx of the lower electrode 23 decreases in the order of the lower electrodes 23-1, 23-2, 23-3, 23-4, 23-5, and 23-6. Arrangement pitches Px of the lower electrodes 23 in the first direction Dx are all equal. That is, a space SP between the adjacent lower electrodes 23 increases in the order of the lower electrodes 23-1, 23-2, 23-3, 23-4, 23-5, and 23-6.

The organic semiconductor layer 30 is rectangular in plan view and is provided extending across the photodiodes PD (lower electrodes 23).

With such a configuration, the photodiodes PD are formed to have different light-receiving areas depending on the distance from the light sources 53 and 54. The detection sensitivity increases as the distance from the light sources 53 and 54 increases, and decreases as the distance from the light sources 53 and 54 decreases. Therefore, the detection device 1 can reduce variations in detection sensitivity of the photodiodes PD due to the distance from the light sources 53 and 54. In other words, even the photodiode PD1 disposed farther from the light sources 53 and 54 can capture an amount of the light L1 sufficient to detect the biometric information. Alternatively, the photodiode PD6 disposed closer to the light sources 53 and 54 can be restrained from capturing an excessive amount of the light L1 that is more than an amount of light required to detect the biometric information.

Although six photodiodes PD are arranged in FIG. 1, this is merely an example and can be changed as appropriate. Two to five photodiodes PD may be arranged, or seven or more photodiodes PD may be arranged. Although the light-receiving areas of the photodiodes PD1 to PD6 are formed to gradually become smaller, the present disclosure is not limited to this configuration. The adjacent photodiodes PD may have an equal light-receiving area.

The following describes a multilayer configuration of the photodiode PD. FIG. 3 is a sectional view along III-III′ of FIG. 1.

In the following description, a direction from the substrate 21 toward a sealing film 28 in a direction orthogonal to a surface of the substrate 21 is referred to as “upper side” or simply “above”. A direction from the sealing film 28 toward the substrate 21 is referred to as “lower side” or simply “below”.

As illustrated in FIG. 3, the substrate 21 is an insulating substrate and is made using, for example, glass or a resin material. The substrate 21 is not limited to having a flat plate shape and may have a curved surface. In this case, the substrate 21 may be made of a film-like resin.

An insulating film 27 is provided on the substrate 21. The insulating film 27 may be an inorganic insulating film or an organic insulating film. The photodiode PD is provided on the insulating film 27. In more detail, the photodiode PD includes the lower electrode 23, the lower buffer layer 32, the active layer 31, the upper buffer layer 33, and the upper electrode 24. In the photodiode PD, the lower electrode 23, the lower buffer layer 32 (hole transport layer), the active layer 31, the upper buffer layer 33 (electron transport layer), and the upper electrode 24 are stacked in this order in the direction orthogonal to the substrate 21.

The lower electrode 23 is an anode electrode of the photodiode PD, and is formed of, for example, a light-transmitting conductive material such as indium tin oxide (ITO).

The active layer 31 changes in characteristics (for example, voltage-current characteristics and resistance value) according to light emitted thereto. An organic material is used as a material of the active layer 31. Specifically, the active layer 31 has a bulk heterostructure containing a mixture of a p-type organic semiconductor and an n-type fullerene derivative (PCBM) that is an n-type organic semiconductor. As the active layer 31, low-molecular-weight organic materials can be used including, for example, fullerene (C60), phenyl-C61-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 31 can be formed by a vapor deposition process (dry process) using any of the low-molecular-weight organic materials listed above. In this case, the active layer 31 may be, for example, a multilayered film of CuPc and F₁₆CuPc, or a multilayered film of rubrene and C₆₀. The active layer 31 can also be formed by a coating process (wet process). In this case, the active layer 31 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 31 can be a film made of a mixture of P3HT and PCBM, or a film made of a mixture of F8BT and PDI.

The lower buffer layer 32 is a hole transport layer, and the upper buffer layer 33 is an electron transport layer. The lower buffer layer 32 and the upper buffer layer 33 are provided to facilitate holes and electrons generated in the active layer 31 to reach the lower electrode 23 or the upper electrode 24. The lower buffer layer 32 (hole transport layer) is in direct contact with the top of the lower electrode 23 and is also provided in areas between the adjacent lower electrodes 23. The active layer 31 is in direct contact with the top of the lower buffer layer 32. The material of the hole transport layer is a metal oxide layer. For example, tungsten oxide (WO₃) or molybdenum oxide is used as the metal oxide layer. The upper buffer layer 33 (electron transport layer) is in direct contact with the top of the active layer 31, and the upper electrode 24 is in direct contact with the top of the upper buffer layer 33. Polyethylenimine ethoxylated (PEIE) is used as a material of the electron transport layer.

The materials and the manufacturing methods of the lower buffer layer 32, the active layer 31, and the upper buffer layer 33 are merely exemplary, and other materials and manufacturing methods may be used. For example, each of the lower buffer layer 32 and the upper buffer layer 33 is not limited to a single-layer film, but may be formed as a multilayered film that includes an electron block layer and a hole block layer.

The upper electrode 24 is provided on the upper buffer layer 33. The upper electrode 24 is a cathode electrode of the photodiode PD and is continuously formed over the entire detection area AA. In other words, the upper electrode 24 is continuously provided as the top layer of the photodiodes PD. The upper electrode 24 faces the lower electrodes 23 with the lower buffer layer 32, the active layer 31, and the upper buffer layer 33 interposed therebetween. The upper electrode 24 is formed of, for example, a light-transmitting conductive material such as ITO or indium zinc oxide (IZO). The detection device 1 of the present embodiment is formed as a top-surface light-receiving optical sensor as described with reference to FIG. 2. The detection device 1 is not limited to this configuration and may be formed as a bottom-surface light-receiving optical sensor in which the light from the object to be detected passes through the substrate 21 and enters the photodiode PD.

The sealing film 28 is provided on the upper electrode 24. An inorganic insulating film such as a silicon nitride film or an aluminum oxide film or a resin film such as an acrylic film is used as the sealing film 28. The sealing film 28 is not limited to a single layer, and may be a multilayered film having two or more layers obtained by combining the inorganic film with the resin film mentioned above. The sealing film 28 well seals the photodiode PD and thus can reduce water entering the photodiode PD from the upper surface side thereof.

### First Modification of First Embodiment

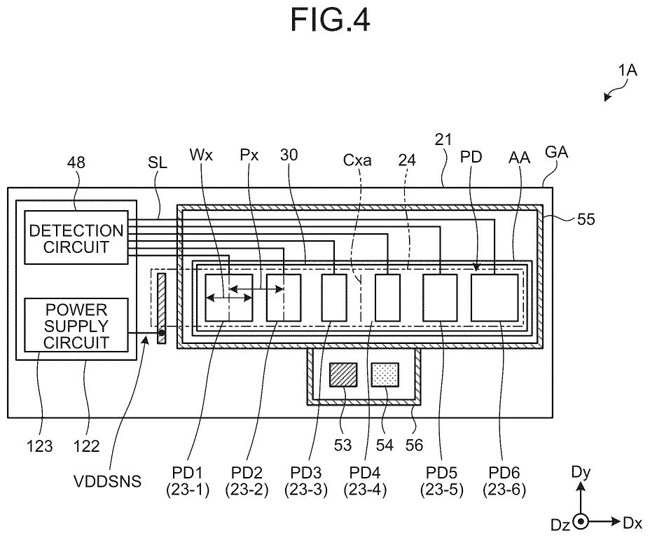

FIG. 4 is a plan view illustrating a detection device according to a first modification of the first embodiment. In the following description, the same components as those described in the embodiment above are denoted by the same reference numerals, and the description thereof will not be repeated.

As illustrated in FIG. 4, a detection device 1A according to the first modification differs from the detection device of the first embodiment described above in the locations of the light sources 53 and 54. Specifically, the light sources 53 and 54 are arranged adjacent to the photodiodes PD in the second direction Dy and adjacent to a central portion of the photodiodes PD arranged in the first direction Dx in the peripheral area GA outside the detection area AA.

That is, the photodiodes PD3 and PD4 disposed in the central portion in the first direction Dx among the photodiodes PD have shorter distances from the light sources 53 and 54. The photodiodes PD1 and PD6 disposed at opposite ends in the first direction Dx have longer distances from the light sources 53 and 54. The photodiodes PD2 and PD5 are disposed so as to have intermediate distances from the light sources 53 and 54 longer than those of the photodiodes PD3, PD4 and shorter than those of the photodiodes PD1, PD6.

In the first modification, the light-receiving areas of the photodiodes PD1, PD2, and PD3 are line-symmetrical to the light-receiving areas of the photodiodes PD4, PD5, and PD6 with respect to a sensor central axis Cxa as an axis of symmetry. The light-receiving area of the photodiode PD1 is equal to that of the photodiode PD6. The light-receiving area of the photodiode PD2 is equal to that of the photodiode PD5. The light-receiving area of the photodiode PD3 is equal to that of the photodiode PD4.

The sensor central axis Cxa is an imaginary line that extends in a direction parallel to the second direction Dy and passes through a midpoint of an imaginary line connecting the geometric centers of the photodiodes PD1 and PD6 disposed at opposite ends in the first direction Dx among the photodiodes PD.

The light-receiving area of the photodiode PD increases as the distance from the light sources 53 and 54 increases. That is, on the left side of the sensor central axis Cxa in FIG. 4, the light-receiving area increases in the order of the photodiodes PD3, PD2, and PD1 as the distance from the light sources 53 and 54 increases. On the right side of the sensor central axis Cxa in FIG. 4, the light-receiving area increases in the order of the photodiodes PD4, PD5, and PD6.

In more detail, the light-receiving area of the photodiode PD1 (first photodiode) is larger than that of the photodiode PD2 (second photodiode) having a shorter distance from the light sources 53 and 54 than that of the photodiode PD1. Similarly, the light-receiving area of the photodiode PD2 is larger than that of the photodiode PD3 (third photodiode) having a shorter distance from the light sources 53 and 54 than that of the photodiode PD2.

Furthermore, the light-receiving area of the photodiode PD6 (sixth photodiode) is larger than that of the photodiode PD5 (fifth photodiode) having a shorter distance from the light sources 53 and 54 than that of the photodiode PD6. Similarly, the light-receiving area of the photodiode PD5 is larger than that of the photodiode PD4 (fourth photodiode) having a shorter distance from the light sources 53 and 54 than that of the photodiode PD5.

The areas of the lower electrodes 23-1, 23-2, and 23-3 are line-symmetrical to the areas of the lower electrodes 23-4, 23-5, and 23-6 with respect to the sensor central axis Cxa as the axis of symmetry. The area of the lower electrode 23-1 is equal to that of the lower electrode 23-6. The area of the lower electrode 23-2 is equal to that of the lower electrode 23-5. The area of the lower electrode 23-3 is equal to that of the lower electrode 23-4.

In the first modification, on the left side of the sensor central axis Cxa in FIG. 4, the area of the lower electrode 23 increases in the order of the lower electrodes 23-3, 23-2, and 23-1. That is, the area of the lower electrode 23-1 (sensor electrode) of the photodiode PD1 (first photodiode) is larger than that of the lower electrode 23-2 (sensor electrode) of the photodiode PD2 (second photodiode) having a shorter distance from the light sources 53 and 54 than that of the photodiode PD1. The area of the lower electrode 23-2 (sensor electrode) of the photodiode PD2 is larger than that of the lower electrode 23-3 (sensor electrode) of the photodiode PD3 (third photodiode) having a shorter distance from the light sources 53 and 54 than that of the photodiode PD2.

On the right side of the sensor central axis Cxa in FIG. 4, the area of the lower electrode 23 increases in the order of the lower electrodes 23-4, 23-5, and 23-6. That is, the area of the lower electrode 23-6 (sensor electrode) of the photodiode PD6 (sixth photodiode) is larger than that of the lower electrode 23-5 (sensor electrode) of the photodiode PD5 (fifth photodiode) having a shorter distance from the light sources 53 and 54 than that of the photodiode PD6. The area of the lower electrode 23-5 (sensor electrode) of the photodiode PD5 is larger than that of the lower electrode 23-4 (sensor electrode) of the photodiode PD4 (fourth photodiode) having a shorter distance from the light sources 53 and 54 than that of the photodiode PD5.

In more detail, the width Wx in the first direction Dx of the lower electrode 23 decreases in the order of the lower electrodes 23-1, 23-2, and 23-3. The width Wx in the first direction Dx of the lower electrode 23 decreases in the order the lower electrodes 23-6, 23-5, and 23-4. The arrangement pitches Px of the lower electrodes 23 in the first direction Dx are all equal. That is, the space SP between the adjacent lower electrodes 23 increases in the order of the lower electrodes 23-1, 23-2, 23-3 and increases in the order of the lower electrodes 23-6, 23-5, and 23-4.

In this way, even when the arrangement relation between the photodiodes PD and the light sources 53 and 54 is changed, the variations in detection sensitivity can be reduced by varying the light-receiving areas of the photodiodes PD depending on the distances from the light sources 53 and 54. Specifically, in the first modification, even the photodiodes PD1 and PD6 disposed farther from the light sources 53 and 54 can receive the light L1 sufficient to detect the biometric information. Alternatively, the photodiodes PD3 and PD4 disposed closer to the light sources 53 and 54 can be restrained from capturing an excessive amount of the light L1 in the detection of biometric information.

### Second Modification of First Embodiment

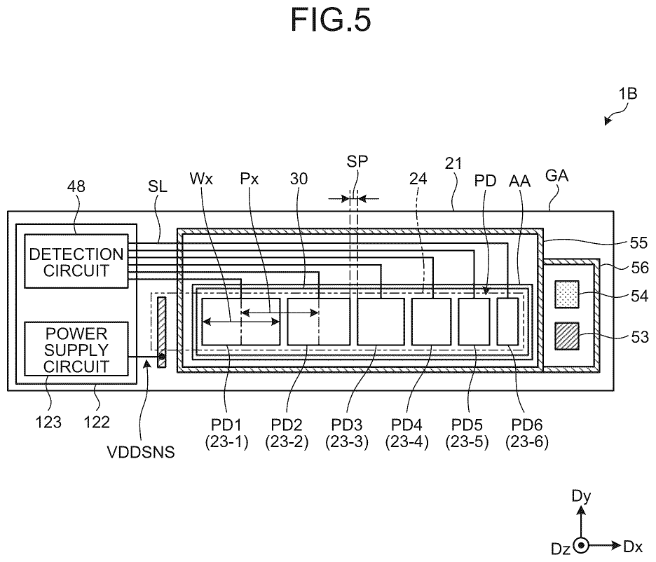

FIG. 5 is a plan view illustrating a detection device according to a second modification of the first embodiment. As illustrated in FIG. 5, in a detection device 1B of the second modification, the positional relation between the photodiodes PD and the light sources 53 and 54 is the same as that in the first embodiment (refer to FIG. 1) described above. The configuration of the detection device 1B of the second modification differs from that in the first embodiment in that the spaces SP between the adjacent lower electrodes 23 have the same size.

In the second modification, the photodiodes PD are formed such that: the photodiode PD, which has a longer distance from the light sources 53 and 54, has a larger light-receiving area; and the photodiode PD, which has a shorter distance from the light sources 53 and 54, has a smaller light-receiving area. Specifically, the light-receiving area decreases in the order of the photodiodes PD1, PD2, PD3, PD4, PD5, and PD6.

The width Wx in the first direction Dx of the lower electrode 23 decreases in the order of the lower electrodes 23-1, 23-2, 23-3, 23-4, 23-5, and 23-6. The spaces SP between the adjacent lower electrodes 23 of the lower electrodes 23-1, 23-2, 23-3, 23-4, 23-5, and 23-6 are set to be equal to one another. That is, the arrangement pitch Px of the lower electrodes 23 in the first direction Dx decreases as the distance from the light sources 53 and 54 decreases.

As a result, in the second modification, the light-receiving areas of the photodiodes PD, that is, the areas of the lower electrodes 23 are formed to be larger than those in the first embodiment described above. Therefore, compared with the first embodiment described above, the amount of the light L1 incident on each of the photodiodes PD can be increased, and the detection sensitivity can be improved.

### Third Modification of First Embodiment

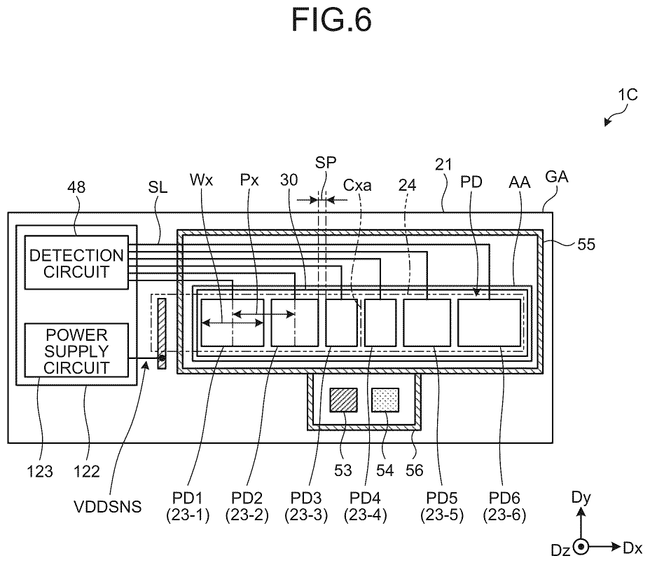

FIG. 6 is a plan view illustrating a detection device according to a third modification of the first embodiment. As illustrated in FIG. 6, in a detection device 1C of the third modification, the positional relation between the photodiodes PD and the light sources 53 and 54 is the same as that in the first modification (refer to FIG. 4) described above. The configuration of the detection device 1C of the third modification differs from that in the first modification in that the spaces SP between the adjacent lower electrodes 23 have the same size.

In the third modification, the light-receiving areas of the photodiodes PD1, PD2, and PD3 are line-symmetrical to the light-receiving areas of the photodiodes PD4, PD5, and PD6 with respect to the sensor central axis Cxa as the axis of symmetry. That is, the areas of the lower electrodes 23-1, 23-2, and 23-3 are line-symmetrical to the areas of the lower electrodes 23-4, 23-5, and 23-6 with respect to the sensor central axis Cxa as the axis of symmetry. The area of the lower electrode 23-1 is equal to that of the lower electrode 23-6. The area of the lower electrode 23-2 is equal to that of the lower electrode 23-5. The area of the lower electrode 23-3 is equal to that of the lower electrode 23-4.

In more detail, the width Wx in the first direction Dx of the lower electrode 23 decreases in the order of the lower electrodes 23-1, 23-2, and 23-3 as the distance from the light sources 53 and 54 decreases. The width Wx in the first direction Dx of the lower electrode 23 decreases in the order of the lower electrodes 23-6, 23-5, and 23-4 as the distance from the light sources 53 and 54 decreases. The spaces SP between the adjacent lower electrodes 23 are all equal. That is, the arrangement pitch Px in the first direction Dx of the adjacent lower electrodes 23 decreases in the order of the lower electrodes 23-1, 23-2, and 23-3, and decreases in the order of the lower electrodes 23-6, 23-5, and 23-4.

With this configuration of the third modification, the light-receiving areas of the photodiodes PD, that is, the areas of the lower electrodes 23 are formed to be larger than those in the first modification described above. Therefore, in the third modification, compared with the first modification described above, the amount of the light L1 incident on the photodiodes PD can be increased, and the detection sensitivity can be improved.

### Second Embodiment

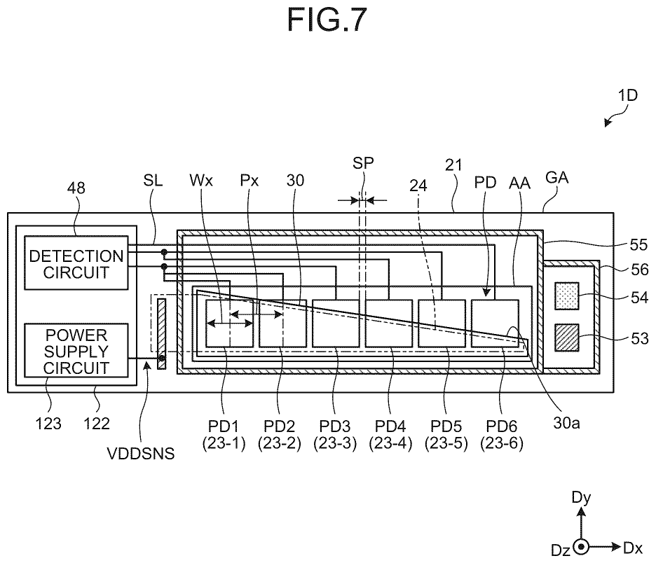

FIG. 7 is a plan view illustrating a detection device according to a second embodiment. As illustrated in FIG. 7, in a detection device 1D of the second embodiment, the positional relation between the photodiodes PD and the light sources 53 and 54 is the same as that in the first embodiment (refer to FIG. 1) described above.

As illustrated in FIG. 7, in the detection device 1D of the second embodiment, the organic semiconductor layer 30 has a substantially triangular shape in plan view. That is, one side of the organic semiconductor layer 30 provided extending across the photodiodes PD has an inclined portion 30a. In plan view, the inclined portion 30a of the organic semiconductor layer 30 extends so as to overlap the lower electrodes 23 of the photodiodes PD. The width in the second direction Dy of the organic semiconductor layer 30 continuously decreases from photodiode PD1 toward the photodiode PD6, that is, as the distance from the light sources 53 and 54 becomes shorter.

In the present embodiment, the shape and area of the lower electrodes 23 are set to be equal to one another. That is, the lower electrodes 23 are all rectangular in shape; and the width Wx, the arrangement pitch Px, and the space SP in the first direction Dx are each set to be equal from the lower electrode 23-1 to the lower electrode 23-6.

With such a configuration, an overlapping area where the lower electrode 23-1 (sensor electrode) of the photodiode PD1 (first photodiode) and the organic semiconductor layer 30 overlap each other is larger than an overlapping area where the lower electrode 23-2 of the photodiode PD2 (second photodiode) and the organic semiconductor layer 30 overlap each other, wherein the photodiode PD2 has a shorter distance from the light sources 53 and 54 than that of the photodiode PD1. The overlapping area where the lower electrode 23-2 (sensor electrode) of the photodiode PD2 and the organic semiconductor layer 30 overlap each other is larger than an overlapping area where the lower electrode 23-3 of the photodiode PD3 (third photodiode) and the organic semiconductor layer 30 overlap each other, wherein the photodiode PD3 has a shorter distance from the light sources 53 and 54 than that of the photodiode PD2. Similarly, the area where the lower electrode 23 and the organic semiconductor layer 30 overlap each other decreases in the order of the photodiodes PD1, PD2, PD3, PD4, PD5, and PD6.

With such a configuration, the light-receiving areas of the photodiodes PD1, PD2, PD3, PD4, PD5, and PD6 are formed to be smaller in this order. That is, the light-receiving area of the photodiode PD increases as the distance from the light sources 53 and 54 increases, and the light-receiving area of the photodiode PD decreases as the distance from the light sources 53 and 54 decreases. As a result, in the third modification, the variations in detection sensitivity of the photodiodes PD due to the distance from the light sources 53 and 54 can be reduced.

### Fourth Modification of Second Embodiment

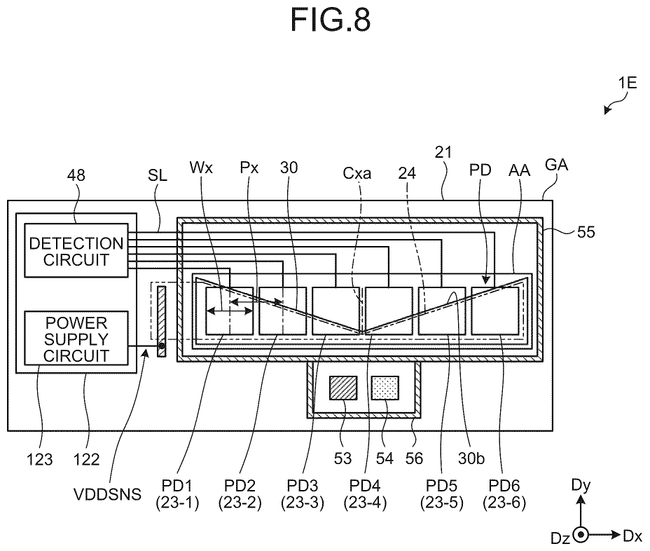

FIG. 8 is a plan view illustrating a detection device according to a fourth modification of the second embodiment. As illustrated in FIG. 8, in a detection device 1E of the fourth modification of the second embodiment, the positional relation between the photodiodes PD and the light sources 53 and 54 is the same as that in the first modification (refer to FIG. 4) described above.

As illustrated in FIG. 8, in the detection device 1E according to the fourth modification of the second embodiment, one side of the organic semiconductor layer 30 provided extending across the photodiodes PD has a recess 30b in plan view. In plan view, the side of the organic semiconductor layer 30 forming the recess 30b extends so as to overlap the lower electrodes 23 of the photodiodes PD. The width in the second direction Dy of the organic semiconductor layer 30 is larger at both ends in the first direction Dx and decreases toward the sensor central axis Cxa. That is, the width in the second direction Dy of the organic semiconductor layer 30 continuously decreases from the photodiodes PD1 and PD6, which have longer distances from the light sources 53 and 54, toward the photodiodes PD3 and PD4, which have shorter distances from the light sources 53 and 54.

An overlapping area where the lower electrode 23-1 (sensor electrode) of the photodiode PD1 (first photodiode) and the organic semiconductor layer 30 overlap each other is larger than an overlapping area where the lower electrode 23-2 of the photodiode PD2 (second photodiode) and the organic semiconductor layer 30 overlap each other, wherein the photodiode PD2 has a shorter distance from the light sources 53 and 54 than that of the photodiode PD1. The overlapping area where the lower electrode 23-2 (sensor electrode) of the photodiode PD2 and the organic semiconductor layer 30 overlap each other is larger than an overlapping area where the lower electrode 23-3 of the photodiode PD3 (third photodiode) and the organic semiconductor layer 30 overlap each other, wherein the photodiode PD3 has a shorter distance from the light sources 53 and 54 than that of the photodiode PD2.

An overlapping area where the lower electrode 23-6 (sensor electrode) of the photodiode PD6 (sixth photodiode) and the organic semiconductor layer 30 overlap each other is larger than an overlapping area where the lower electrode 23-5 of the photodiode PD5 (fifth photodiode) and the organic semiconductor layer 30 overlap each other, wherein the photodiode PD5 has a shorter distance from the light sources 53 and 54 than that of the photodiode PD6. The overlapping area where the lower electrode 23-5 (sensor electrode) of the photodiode PD5 and the organic semiconductor layer 30 overlap each other is larger than an overlapping area where the lower electrode 23-4 of the photodiode PD4 (fourth photodiode) and the organic semiconductor layer 30 overlap each other, wherein the photodiode PD4 has a shorter distance from the light sources 53 and 54 than that of the photodiode PD5.

With such a configuration, the light-receiving areas of the photodiodes PD1, PD2, and PD3 are line-symmetrical to the light-receiving areas of the photodiodes PD4, PD5, and PD6 with respect to the sensor central axis Cxa as the axis of symmetry. On the left side of the sensor central axis Cxa, the light-receiving area (overlapping area) increases in the order of the photodiodes PD3, PD2, and PD1 as the distance from the light sources 53 and 54 increases. On the right side of the sensor central axis Cxa, the light-receiving area (overlapping area) increases in the order of the photodiodes PD4, PD5, and PD6. As a result, in the fourth modification, the variations in detection sensitivity of the photodiodes PD due to the distance from the light sources 53 and 54 can be reduced.

In the second embodiment and the fourth modification, the lower electrodes 23 have the same shape and area, but the present disclosure is not limited to this configuration. The second embodiment and the fourth modification may be combined with any of the first embodiment and the first to the third modifications as appropriate. That is, in the second embodiment and the fourth modification, the shape of the organic semiconductor layer 30 may be changed and the shape and area of the lower electrodes 23 of the photodiodes PD may be changed depending on the distance from the light sources 53 and 54.

### Third Embodiment

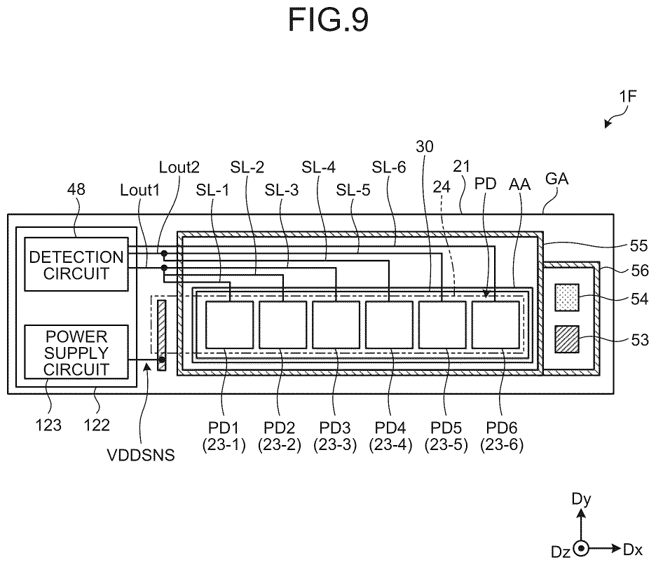

FIG. 9 is a plan view illustrating a detection device according to a third embodiment. As illustrated in FIG. 9, in a detection device 1F of the third embodiment, the positional relation between the photodiodes PD and the light sources 53 and 54 is the same as that in the first embodiment (refer to FIG. 1) described above. The detection device 1F of the third embodiment differs from the detection device in each of the embodiments and the modifications described above in coupling structure between the photodiodes PD and the detection circuit 48.

As illustrated in FIG. 9, signal lines SL-1, SL-2, and SL-3 coupled to the photodiodes PD1, PD2, and PD3, respectively, are coupled to the detection circuit 48 through a common output signal line Lout1. That is, the photodiodes PD1, PD2, and PD3 having longer distances from the light sources 53 and 54 than those of the photodiodes PD4, PD5, and PD6 are bundled together as a single sensor element and coupled to the detection circuit 48.

Signal lines SL-4 and SL-5 coupled to the photodiodes PD4 and PD5, respectively, are coupled to the detection circuit 48 through a common output signal line Lout2. That is, the photodiodes PD4 and PD5 having longer distances from the light sources 53 and 54 than that of the photodiode PD6 are bundled together as a single sensor element and coupled to the detection circuit 48. The photodiode PD6 having a shorter distance from the light sources 53 and 54 is coupled to the detection circuit 48 through one signal line SL-6.

In the present embodiment, the shapes and areas of the lower electrodes 23 are set to be equal to one another. That is, the width Wx, the arrangement pitch Px, and the space SP in the first direction Dx of the lower electrodes 23 are each set to be equal from the lower electrode 23-1 to the lower electrode 23-6. The organic semiconductor layer 30 is rectangular in plan view and is provided extending across the photodiodes PD. Although one detection circuit 48 is provided, a plurality of the detection circuits 48 may be provided, and, for example, the detection circuits 48 may be respectively provided for the bundles of the coupled photodiodes PD.

Thus, in the third embodiment, the photodiodes PD are bundled based on the distance from the light sources 53 and 54 and coupled to the detection circuit 48. Specifically, the total light-receiving area of the photodiodes PD1, PD2, and PD3 is substantially three times the light-receiving area of the photodiode PD6. The total light-receiving area of the photodiodes PD4 and PD5 is substantially twice the light-receiving area of the photodiode PD6. As a result, the photodiodes PD are formed such that the total light-receiving area of the photodiodes PD is substantially larger as the distance from the light sources 53 and 54 is longer, and substantially smaller as the distance from the light sources 53 and 54 is shorter.

### Fifth Modification of Third Embodiment

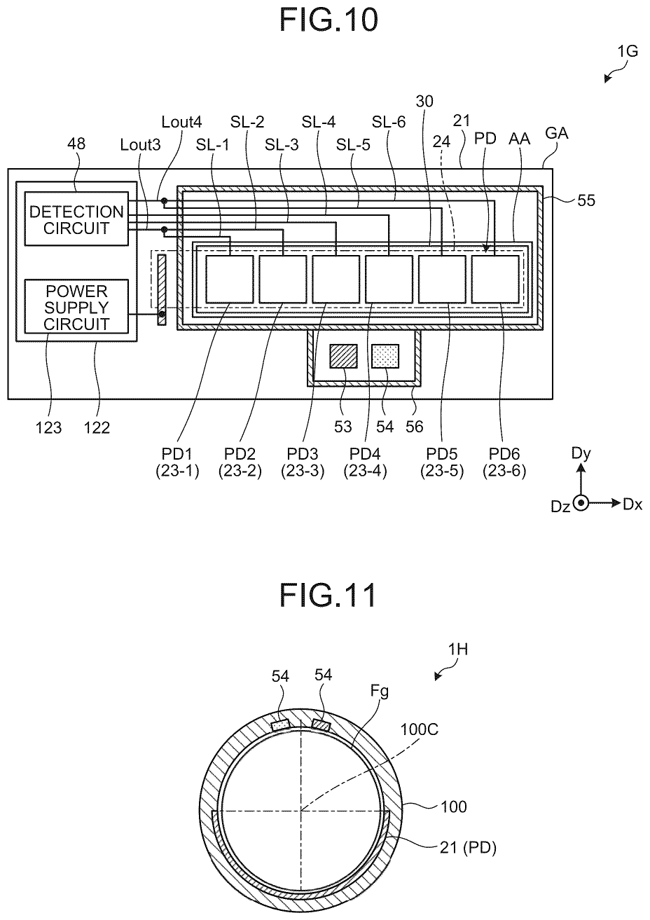

FIG. 10 is a plan view illustrating a detection device according to a fifth modification of the third embodiment. As illustrated in FIG. 10, in a detection device 1G of the fifth modification of the third embodiment, the positional relation between the photodiodes PD and the light sources 53 and 54 is the same as that in the first modification (refer to FIG. 4) described above.

As illustrated in FIG. 10, the signal lines SL-1 and SL-2 coupled to the photodiodes PD1 and PD2, respectively, are coupled to the detection circuit 48 through a common output signal line Lout3. That is, the photodiodes PD1 and PD2 having longer distances from the light sources 53 and 54 than that of the photodiode PD3 are bundled together as a single sensor element and coupled to the detection circuit 48. The photodiode PD3 having a shorter distance from the light sources 53 and 54 than those of the photodiodes PD1 and PD2 is coupled to the detection circuit 48 through one signal line SL-3.

The signal lines SL-5 and SL-6 coupled to the photodiodes PD5 and PD6, respectively, are coupled to the detection circuit 48 through a common output signal line Lout4. That is, the photodiodes PD5 and PD6 having longer distances from the light sources 53 and 54 than that of the photodiode PD4 are bundled together as a single sensor element and are coupled to the detection circuit 48. The photodiode PD4 having a shorter distance from the light sources 53 and 54 than that of the photodiodes PD5 and PD6 is coupled to the detection circuit 48 through one signal line SL-4.

Thus, in the fifth modification, the photodiodes PD are bundled based on the distance from the light sources 53 and 54, and coupled to the detection circuit 48. Specifically, the total light-receiving area of the photodiodes PD1 and PD2 is substantially twice the light-receiving area of the photodiode PD3. The total light-receiving area of the photodiodes PD5 and PD6 is substantially twice the light-receiving area of the photodiode PD4. In also the fifth modification, the photodiodes PD are formed such that the light-receiving area of the photodiode PD is substantially larger as the distance from the light sources 53 and 54 is longer, and substantially smaller as the distance from the light sources 53 and 54 is shorter.

The third embodiment and the fifth modification may be combined with any of the first embodiment, the second embodiment, and the modifications as appropriate. That is, the coupling between the photodiodes PD and the detection circuit 48 may be varied depending on the distance from the light sources 53 and 54, and in addition, the shape and area of the lower electrodes 23 of the photodiodes PD may be varied, or the shape of the organic semiconductor layer 30 may be varied.

### Fourth Embodiment

FIG. 11 is a sectional view illustrating an exemplary use of a detection device according to a fourth embodiment for measuring the biometric information. As illustrated in FIG. 11, a detection device 1H according to the fourth embodiment includes an annular housing 100. The substrate 21, the photodiodes PD, and the light sources 53 and 54 are provided in the annular housing 100. The substrate 21 is provided so as to be curved along an inner peripheral surface of the annular housing 100, and the photodiodes PD are arranged along the inner peripheral surface of the annular housing 100. The light sources 53, 54 and the photodiodes PD are provided on opposite sides to each other with a center 100C of the annular housing 100 interposed therebetween.

When measuring the biometric information, the annular housing 100 is worn on the object to be detected Fg such as a finger, and the object to be detected Fg is placed between the light sources 53, 54 and the photodiodes PD. The light L1 emitted from the light sources 53 and 54 is scattered and reflected in the object to be detected Fg and enters the photodiodes PD. In the present embodiment, in a direction along the inner periphery of the annular housing 100, the photodiodes PD disposed at opposite ends of the substrate 21 have shorter distances from the light sources 53 and 54, and the photodiodes PD disposed in a central portion of the substrate 21 have a longer distance from the light sources 53 and 54.

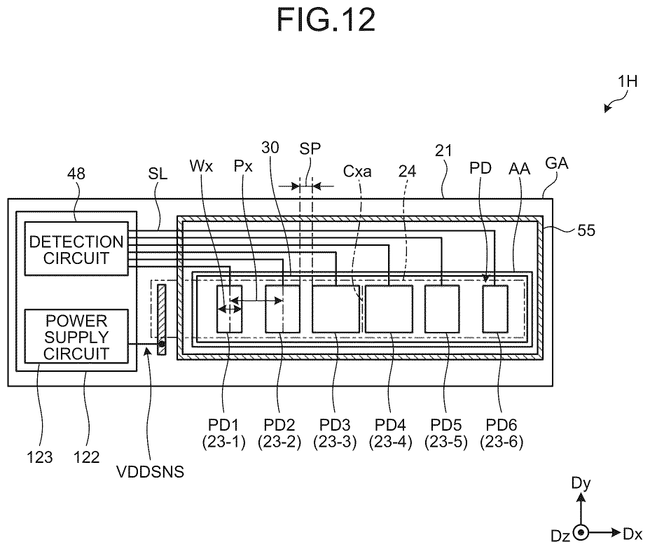

FIG. 12 is a plan view illustrating the detection device according to the fourth embodiment. FIG. 12 illustrates a plan view of the substrate 21 and the photodiodes PD illustrated in FIG. 11 in a state of being developed on a plane. The first direction Dx illustrated in FIG. 12 corresponds to a peripheral direction of the annular housing 100 when the substrate 21 is incorporated in the annular housing 100. Similarly, the second direction Dy corresponds to a direction parallel to the axial direction of the annular housing 100. The third direction Dz corresponds to a radial direction of the annular housing 100.

As illustrated in FIG. 12, in the detection device 1H according to the fourth embodiment, the photodiodes PD1 and PD6 disposed at opposite ends in the first direction Dx have shorter distances from the light sources 53 and 54 (refer to FIG. 11). Among the photodiodes PD, the photodiodes PD3 and PD4 disposed in the central portion in the first direction Dx have longer distances from the light sources 53 and 54. The photodiodes PD2 and PD5 are disposed so as to have intermediate distances from the light sources 53 and 54 longer than those of the photodiodes PD3, PD4 and shorter than those of the photodiodes PD1, PD6.

The light-receiving area increases in the order of the photodiodes PD1, PD2, and PD3 as the distance from the light sources 53 and 54 increases. The light-receiving area also increases in the order of the photodiodes PD6, PD5, and PD4. That is, the areas of the lower electrodes 23-3 and 23-4 (sensor electrodes) of the photodiodes PD3 and PD4 (first photodiodes) located in a central portion in a peripheral direction (first direction Dx) of the detection area AA are larger than the areas of the lower electrodes 23-1 and 23-6 (sensor electrodes) of the photodiodes PD1 and PD6 (second photodiodes) located in the outer edge portions in the peripheral direction (first direction Dx) of the detection area AA.

In the fourth embodiment, the light-receiving areas of the photodiodes PD1, PD2, and PD3 are line-symmetrical to the light-receiving areas of the photodiodes PD4, PD5, and PD6 with respect to the sensor central axis Cxa as the axis of symmetry. That is, the areas of the lower electrodes 23-1, 23-2, and 23-3 are line-symmetrical to the areas of the lower electrodes 23-4, 23-5, and 23-6 with respect to the sensor central axis Cxa as the axis of symmetry. The area of the lower electrode 23-1 is equal to that of the lower electrode 23-6. The area of the lower electrode 23-2 is equal to that of the lower electrode 23-5. The area of the lower electrode 23-3 is equal to that of the lower electrode 23-4.

In more detail, the light-receiving area of the photodiode PD3 is larger than that of the photodiode PD2 having a shorter distance from the light sources 53 and 54 than that of the photodiode PD3. Similarly, the light-receiving area of the photodiode PD2 is larger than that of the photodiode PD1 having a shorter distance from the light sources 53 and 54 than that of the photodiode PD2.

In addition, the light-receiving area of the photodiode PD4 is larger than that of the photodiode PD5 having a shorter distance from the light sources 53 and 54 than that of the photodiode PD4. Similarly, the light-receiving area of the photodiode PD5 is larger than that of the photodiode PD6 having a shorter distance from the light sources 53 and 54 than that of the photodiode PD5.

In the first modification, as illustrated in FIG. 12, the area of the lower electrode 23 decreases in the order of the lower electrodes 23-3, 23-2, and 23-1. That is, the area of the lower electrode 23-3 (sensor electrode) of the photodiode PD3 (third photodiode) is larger than that of the lower electrode 23-2 (sensor electrode) of the photodiode PD2 having a shorter distance from the light sources 53 and 54 than that of the photodiode PD3. The area of the lower electrode 23-2 (sensor electrode) of the photodiode PD2 is larger than that of the lower electrode 23-1 (sensor electrode) of the photodiode PD1 having a shorter distance from the light sources 53 and 54 than that of the photodiode PD2.

The area of the lower electrode 23 decreases in the order of the lower electrodes 23-4, 23-5, and 23-6. That is, the area of the lower electrode 23-4 (sensor electrode) of the photodiode PD4 is larger than that of the lower electrode 23-5 (sensor electrode) of the photodiode PD5 having a shorter distance from the light sources 53 and 54 than that of the photodiode PD4. The area of the lower electrode 23-5 (sensor electrode) of the photodiode PD5 is larger than that of the lower electrode 23-6 (sensor electrode) of the photodiode PD6 having a shorter distance from the light sources 53 and 54 than that of the photodiode PD5.

The width Wx in the first direction Dx of the lower electrode 23 decreases in this order of the lower electrodes 23-3, 23-2, and 23-1. The width Wx in the first direction Dx of the lower electrode 23 decreases in the order of the lower electrodes 23-4, 23-5, and 23-6. The arrangement pitches Px of the lower electrodes 23 in the first direction Dx are all equal. That is, the space SP between the adjacent lower electrodes 23 decreases in the order of the lower electrodes 23-1, 23-2, and 23-3 and decreases in the order of the lower electrodes 23-6, 23-5, and 23-4.

In the fourth embodiment, the photodiodes PD and the light sources 53 and 54 are arranged in the annular housing 100. In this case, the positions and numbers of the photodiodes PD and the light sources 53 and 54 are greatly constrained, resulting in variations in distance between the light sources 53, 54 and the photodiodes PD. Even with such a configuration, the variations in detection sensitivity of the photodiodes PD due to the distance from the light sources 53 and 54 can be reduced by varying the light-receiving area of the photodiodes PD depending on the distance from the light sources 53 and 54.

Although FIG. 11 illustrates the example in which the detection device 1H including the annular housing 100 is worn on a finger, the detection device 1H is not limited to this example. The detection device 1H including the annular housing 100 may be worn on another object to be detected Fg, such as an arm or the wrist.

### Sixth Modification of Fourth Embodiment

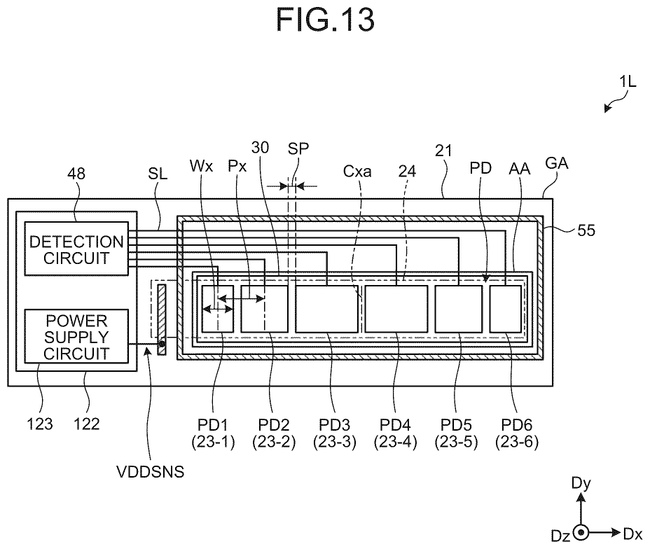

FIG. 13 is a plan view illustrating a detection device according to a sixth modification of the fourth embodiment. In the sixth modification of the fourth embodiment, the positional relation between the photodiodes PD and the light sources 53 and 54 is the same as that in the fourth embodiment described above (refer to FIG. 11). The configuration of a detection device 1I of the sixth modification differs from that in the fourth embodiment in that the spaces SP between the adjacent lower electrodes 23 have the same size.

In the sixth modification, the light-receiving areas of the photodiodes PD1, PD2, and PD3 are line-symmetrical to the light-receiving areas of the photodiodes PD4, PD5, and PD6 with respect to the sensor central axis Cxa as the axis of symmetry. That is, the areas of the lower electrodes 23-1, 23-2, and 23-3 are line-symmetrical to the areas of the lower electrodes 23-4, 23-5, and 23-6 with respect to the sensor central axis Cxa as the axis of symmetry. The area of the lower electrode 23-1 is equal to that of the lower electrode 23-6. The area of the lower electrode 23-2 is equal to that of the lower electrode 23-5. The area of the lower electrode 23-3 is equal to that of the lower electrode 23-4.

In more detail, the width Wx in the first direction Dx of the lower electrode 23 decreases in the order of the lower electrodes 23-3, 23-2, and 23-1, as the distance from the light sources 53 and 54 decreases. The width Wx in the first direction Dx of the lower electrode 23 decreases in the order of the lower electrodes 23-4, 23-5, and 23-6, as the distance from the light sources 53 and 54 decreases. The spaces SP between the adjacent lower electrodes 23 are all equal. That is, the arrangement pitch Px in the first direction Dx of the adjacent lower electrodes 23 decreases in the order of the lower electrodes 23-3, 23-2, and 23-1, and decreases in the order of the lower electrodes 23-4, 23-5, and 23-6.

As a result, in the sixth modification, the light-receiving areas of the photodiodes PD, that is, the areas of the lower electrodes 23 are formed to be larger than those in the fourth embodiment described above. Therefore, compared with the fourth embodiment described above, the amount of the light L1 incident on the photodiodes PD can be increased, and the detection sensitivity can be improved.

### Seventh Modification of Fourth Embodiment

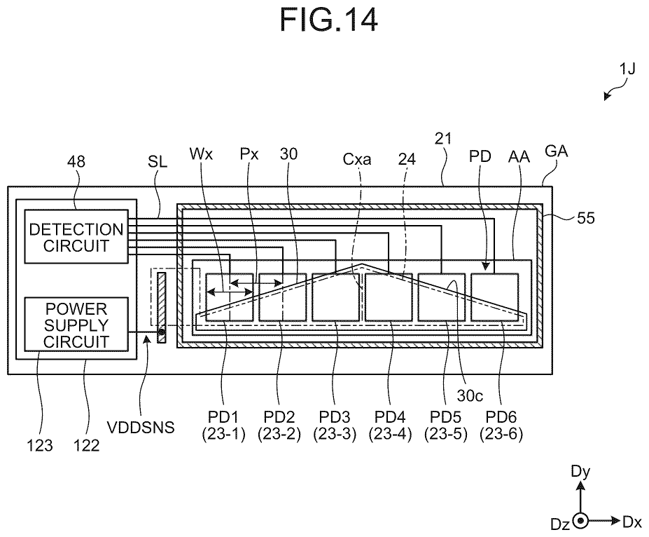

FIG. 14 is a plan view illustrating a detection device according to a seventh modification of the fourth embodiment. In the seventh modification of the fourth embodiment, the positional relation between the photodiodes PD and the light sources 53 and 54 is the same as that in the fourth embodiment described above (refer to FIG. 11).

As illustrated in FIG. 14, in a detection device 1J according to the seventh modification of the fourth embodiment, one side of the organic semiconductor layer 30 provided extending across the photodiodes PD has a protrusion 30c in plan view. In plan view, the side of the organic semiconductor layer 30 forming the protrusion 30c extends so as to overlap the lower electrodes 23 of the photodiodes PD. The width in the second direction Dy of the organic semiconductor layer 30 is larger in the central portion in the peripheral direction (first direction Dx) of the detection area AA and decreases toward the outer edge portions in the peripheral direction (first direction Dx) of the detection area AA. That is, the width in the second direction Dy of the organic semiconductor layer 30 continuously decreases from the photodiodes PD3 and PD4, which have longer distances from the light sources 53 and 54, toward the photodiodes PD1 and PD6, which have shorter distances from the light sources 53 and 54.

An overlapping area where the lower electrodes 23-3 and 23-4 (sensor electrodes) of the photodiodes PD3 and PD4 (first photodiodes) and the organic semiconductor layer 30 overlap each other is larger than an overlapping area where the lower electrodes 23-1 and 23-6 (sensor electrodes) of the photodiodes PD1 and PD6 (second photodiodes) and the organic semiconductor layer 30 overlap each other, wherein the photodiodes PD3 and PD4 are disposed in the central portion in the peripheral direction (first direction Dx) of the detection area AA, and the photodiodes PD1 and PD6 are disposed in the outer edge portions in the peripheral direction (first direction Dx) of the detection area AA.

An overlapping area where the lower electrode 23-3 (sensor electrode) of the photodiode PD3 and the organic semiconductor layer 30 overlap each other is larger than an overlapping area where the lower electrode 23-2 of the photodiode PD2 and the organic semiconductor layer 30 overlap each other, wherein the photodiode PD2 has a shorter distance from the light sources 53 and 54 than that of the photodiode PD3. An overlapping area where the lower electrode 23-2 (sensor electrode) of the photodiode PD2 and the organic semiconductor layer 30 overlap each other is larger than an overlapping area where the lower electrode 23-1 of the photodiode PD1 and the organic semiconductor layer 30 overlap each other, wherein the photodiode PD1 has a shorter distance from the light sources 53 and 54 than that of the photodiode PD2.

An overlapping area where the lower electrode 23-4 (sensor electrode) of the photodiode PD4 and the organic semiconductor layer 30 overlap each other is larger than an overlapping area where the lower electrode 23-5 of the photodiode PD5 and the organic semiconductor layer 30 overlap each other, wherein the photodiode PD5 has a shorter distance than that of the photodiode PD4 from the light sources 53 and 54. An overlapping area wherein the lower electrode 23-5 (sensor electrode) of the photodiode PD5 and the organic semiconductor layer 30 overlap each other is larger than an overlapping area where the lower electrode 23-6 of the photodiode PD6 and the organic semiconductor layer 30 overlap each other, wherein the photodiode PD6 has a shorter distance from the light sources 53 and 54 than that of the photodiode PD5.

With such a configuration, the light-receiving areas of the photodiodes PD1, PD2, and PD3 are line-symmetrical to the light-receiving areas of the photodiodes PD4, PD5, and PD6 with respect to the sensor central axis Cxa as the axis of symmetry. On the left side of the sensor central axis Cxa, the light-receiving area (overlapping area) increases in the order of the photodiodes PD1, PD2, and PD3 as the distance from the light sources 53 and 54 increases. On the right side of the sensor central axis Cxa, the light-receiving area (overlapping area) increases in the order of the photodiodes PD6, PD5, and PD4. Such a configuration can reduce the variations in detection sensitivity of the photodiodes PD due to the distance from the light sources 53 and 54.

In the present embodiment, the shapes and areas of the lower electrodes 23 are set to be equal to one another. That is, the width Wx, the arrangement pitch Px, and the space SP in the first direction Dx of the lower electrodes 23 are each set to be equal from the lower electrode 23-1 to the lower electrode 23-6. However, the seventh modification is not limited to this configuration and can be combined with the configurations of the fourth embodiment and the sixth modification described above. That is, depending on the distance from the light sources 53 and 54, the shape of the organic semiconductor layer 30 may be varied, and in addition, the shape and area of the lower electrodes 23 of the photodiodes PD may be varied.

### Fifth Embodiment

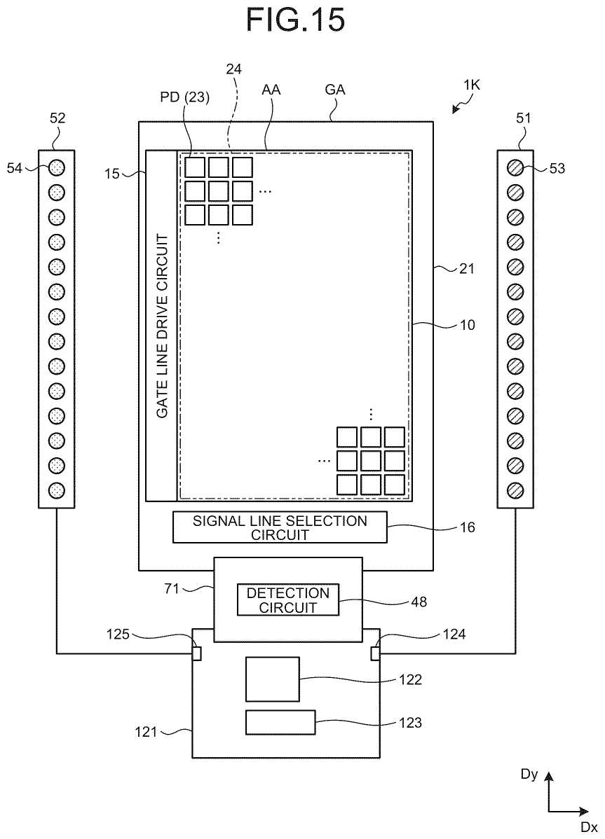

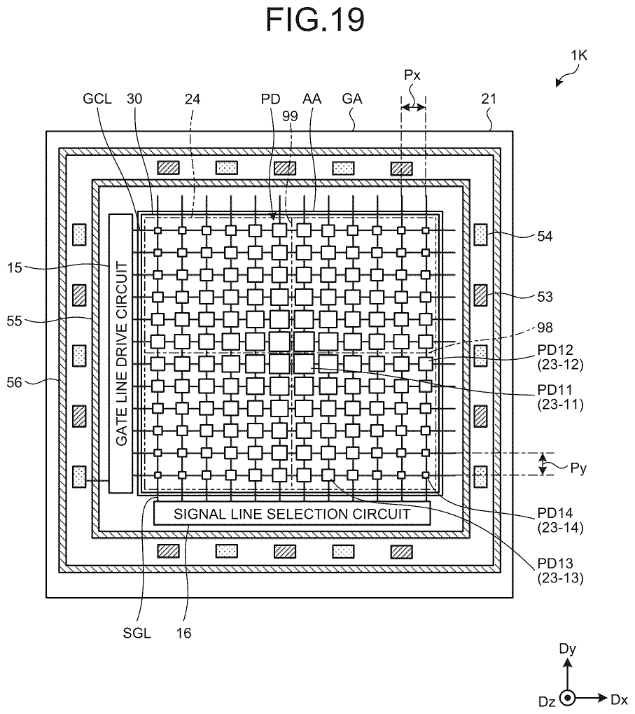

FIG. 15 is a plan view illustrating a detection device according to a fifth embodiment. As illustrated in FIG. 15, a detection device 1K according to the fifth embodiment includes the substrate 21, a sensor 10, a gate line drive circuit 15, a signal line selection circuit 16, the detection circuit 48, the control circuit 122, the power supply circuit 123, a first light source base member 51, a second light source base member 52, and the light sources 53 and 54. The first light source base member 51 is provided with a plurality of the light sources 53. The second light source base member 52 is provided with a plurality of the light sources 54. However, as will be described later with reference to FIG. 19, the light sources 53 and 54 may be provided on the same substrate 21 as the photodiodes PD.

The substrate 21 is electrically coupled to a control substrate 121 through a wiring substrate 71. The wiring substrate 71 is, for example, a flexible printed circuit board or a rigid circuit board. The wiring substrate 71 is provided with the detection circuit 48. The control substrate 121 is provided with the control circuit 122 and the power supply circuit 123. The control circuit 122 is a field-programmable gate array (FPGA), for example. The control circuit 122 supplies control signals to the sensor 10, the gate line drive circuit 15, and the signal line selection circuit 16 to control detection operations of the sensor 10. The control circuit 122 supplies control signals to the light sources 53 and 54 to control lighting and non-lighting of the light sources 53 and 54. The power supply circuit 123 supplies voltage signals including, for example, the sensor power supply signal VDDSNS to the sensor 10, the gate line drive circuit 15, and the signal line selection circuit 16. The power supply circuit 123 supplies a power supply voltage to the light sources 53 and 54.

The photodiodes PD are arranged in a matrix having a row-column configuration in the detection area AA. Each of the photodiodes PD performs detection in response to a gate drive signal VGL supplied from the gate line drive circuit 15. Each of the photodiodes PD outputs, to the signal line selection circuit 16, the electrical signal as the detection signal Vdet corresponding to the light emitted thereto. The detection device 1K detects the information on the object to be detected, based on the detection signals Vdet from the photodiodes PD.

The gate line drive circuit 15 and the signal line selection circuit 16 are provided in the peripheral area GA. Specifically, the gate line drive circuit 15 is provided in an area extending along the second direction Dy in the peripheral area GA. The signal line selection circuit 16 is provided in an area extending along the first direction Dx in the peripheral area GA and is provided between the sensor 10 and the detection circuit 48.

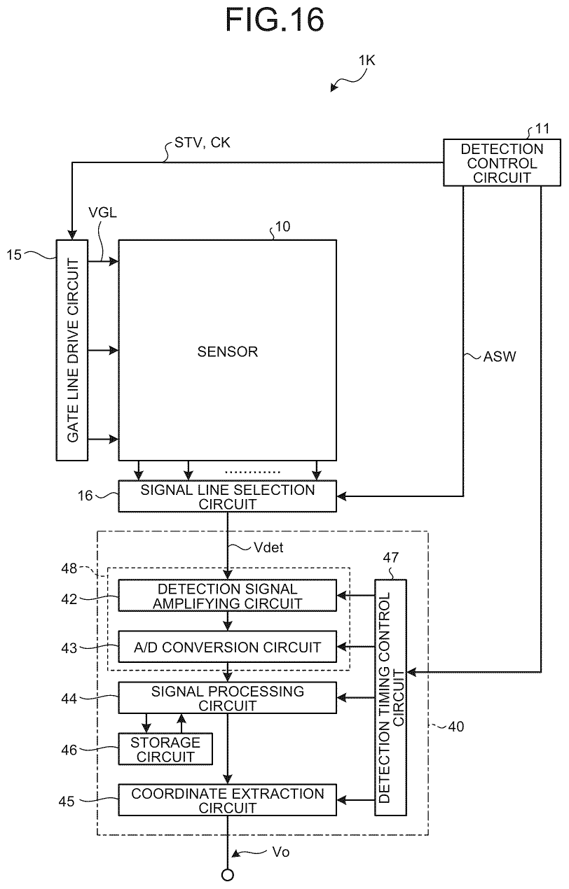

FIG. 16 is a block diagram illustrating a configuration example of the detection device according to the fifth embodiment. As illustrated in FIG. 16, the detection device 1K further includes a detection control circuit 11 and a detector (detection signal processing circuit) 40. The control circuit 122 includes one, some, or all functions of the detection control circuit 11. The control circuit 122 also includes one, some, or all functions of the detector 40 other than those of the detection circuit 48.

The detection control circuit 11 is a circuit that supplies respective control signals to the gate line drive circuit 15, the signal line selection circuit 16, and the detector 40 to control operations of these components. The detection control circuit 11 supplies various control signals including, for example, a start signal STV and a clock signal CK to the gate line drive circuit 15. The detection control circuit 11 also supplies various control signals including, for example, a selection signal ASW to the signal line selection circuit 16. The detection control circuit 11 also supplies various control signals to the light sources 53 and 54 to control the lighting and non-lighting of the respective light sources 53 and 54.

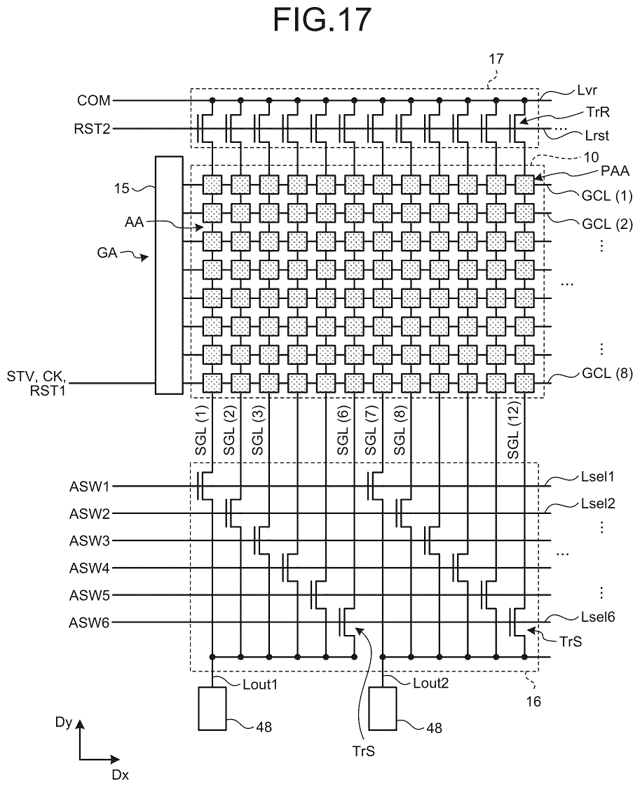

The gate line drive circuit 15 is a circuit that drives a plurality of gate lines GCL (refer to FIG. 17) based on the various control signals. The gate line drive circuit 15 sequentially or simultaneously selects the gate lines GCL and supplies the gate drive signals VGL to the selected gate lines GCL. Through this operation, the gate line drive circuit 15 selects the photodiodes PD coupled to the gate lines GCL.

The signal line selection circuit 16 is a switch circuit that sequentially or simultaneously selects a plurality of signal lines SGL (refer to FIG. 17). The signal line selection circuit 16 is a multiplexer, for example. The signal line selection circuit 16 couples the selected signal lines SGL to the detection circuit 48 based on the selection signal ASW supplied from the detection control circuit 11. Through this operation, the signal line selection circuit 16 outputs the detection signal Vdet of the photodiode PD to the detector 40.

The detector 40 includes the detection circuit 48, a signal processing circuit 44, a coordinate extraction circuit 45, a storage circuit 46, and a detection timing control circuit 47. The detection timing control circuit 47 controls the detection circuit 48, the signal processing circuit 44, and the coordinate extraction circuit 45 to operate them synchronously based on a control signal supplied from the detection control circuit 11.

The detection circuit 48 is an analog front-end (AFE) circuit, for example. The detection circuit 48 is a signal processing circuit having functions of at least a detection signal amplifying circuit 42 and an analog-to-digital (A/D) conversion circuit 43. The detection signal amplifying circuit 42 amplifies the detection signal Vdet. The A/D conversion circuit 43 converts analog signals output from the detection signal amplifying circuit 42 into digital signals.

The signal processing circuit 44 is a logic circuit that detects predetermined physical quantities received by the sensor 10 based on output signals of the detection circuit 48. The signal processing circuit 44 can detect asperities on a surface of a finger or a palm based on the signals from the detection circuit 48 when the finger is in contact with or in proximity to a detection surface. The signal processing circuit 44 can detect the information on the living body based on the signals from the detection circuit 48. Examples of the information on the living body include the vascular image, the pulse waves, the pulsation, and a blood oxygen level of the finger or the palm.

The storage circuit 46 temporarily stores therein signals calculated by the signal processing circuit 44. The storage circuit 46 may be, for example, a random-access memory (RAM) or a register circuit.

The coordinate extraction circuit 45 is a logic circuit that obtains detected coordinates of the asperities on the surface of a finger or the like when the contact or proximity of the finger is detected by the signal processing circuit 44. The coordinate extraction circuit 45 is the logic circuit that also obtains detected coordinates of blood vessels in the finger or the palm. The coordinate extraction circuit 45 combines the detection signals Vdet output from the photodiodes PD of the sensor 10 to generate two-dimensional information representing the shape of the asperities on the surface of the finger or the like and two-dimensional information representing the shape of the blood vessels in the finger or the palm. The coordinate extraction circuit 45 may output an output value from the detection circuit 48 as a sensor output voltage Vo instead of calculating the detected coordinates.

The following describes a circuit configuration example of the detection device 1K. FIG. 17 is a circuit diagram illustrating the detection device according to the fifth embodiment. As illustrated in FIG. 17, the sensor 10 has a plurality of partial detection areas PAA arranged in a matrix having a row-column configuration. Each of the partial detection areas PAA is provided with the photodiode PD.

The gate lines GCL extend in the first direction Dx and are each coupled to the partial detection areas PAA arranged in the first direction Dx. A plurality of gate lines GCL (1), GCL (2), . . . , GCL (8) are arranged in the second direction Dy and are each coupled to the gate line drive circuit 15. In the following description, the gate lines GCL (1), GCL (2), . . . , GCL (8) will each be simply referred to as the gate line GCL when they need not be distinguished from one another. To facilitate understanding of the description, FIG. 17 illustrates eight gate lines GCL. However, this is merely an example, and M gate lines GCL may be arranged (where M is 8 or larger, and is, for example, 256).

The signal lines SGL extend in the second direction Dy and are each coupled to the photodiodes PD in the partial detection areas PAA arranged in the second direction Dy. A plurality of signal lines SGL (1), SGL (2), . . . , SGL (12) are arranged in the first direction Dx and are each coupled to the signal line selection circuit 16 and a reset circuit 17. In the following description, the signal lines SGL (1), SGL (2), . . . , SGL (12) will each be simply referred to as the signal line SGL when they need not be distinguished from one another.

To facilitate understanding of the description, 12 signal lines SGL are illustrated. However, this is merely an example, and N signal lines SGL may be arranged (where N is 12 or larger and is, for example, 252). The resolution of the sensor is, for example, 508 dots per inch (dpi), and the number of cells is 252×256. In FIG. 17, the sensor 10 is provided between the signal line selection circuit 16 and the reset circuit 17. The present disclosure is not limited to this configuration. The signal line selection circuit 16 and the reset circuit 17 may be coupled to ends of the signal lines SGL on the same side.

The gate line drive circuit 15 receives various control signals including, for example, the start signal STV, the clock signal CK, and a reset signal RST1 from the control circuit 122 (refer to FIG. 15). The gate line drive circuit 15 sequentially selects the gate lines GCL (1), GCL (2), . . . , GCL (8) in a time-division manner based on the various control signals. The gate line drive circuit 15 supplies the gate drive signal VGL to the selected one of the gate lines GCL. This operation supplies the gate drive signal VGL to a plurality of drive transistors Tr coupled to the gate line GCL, and corresponding partial detection areas PAA arranged in the first direction Dx are selected as detection targets.

The signal line selection circuit 16 includes a plurality of selection signal lines Lsel, a plurality of output signal lines Lout, and a plurality of output transistors TrS. The output transistors TrS are provided correspondingly to the signal lines SGL. Each of the output transistors TrS is a switch that switches the coupling between one output signal line Lout and one signal line SGL. Six signal lines SGL (1), SGL (2), . . . , SGL (6) are coupled to the common output signal line Lout1. Six signal lines SGL (7), SGL (8), . . . , SGL (12) are coupled to the common output signal line Lout2. The output signal lines Lout1 and Lout2 are each coupled to the detection circuit 48.

The signal lines SGL (1), SGL (2), . . . , SGL (6) are grouped into a first signal line block, and the signal lines SGL (7), SGL (8), . . . , SGL (12) are grouped into a second signal line block. The selection signal lines Lsel are coupled to the gates of the respective output transistors Trs included in one of the signal line blocks. One of the selection signal lines Lsel is coupled to the gates of the output transistors TrS in the signal line blocks.

The control circuit 122 (refer to FIG. 15) sequentially supplies the selection signal ASW to the selection signal lines Lsel. This operation causes the signal line selection circuit 16 to operate the output transistors TrS to sequentially select the signal lines SGL in each of the signal line blocks in a time-division manner. The signal line selection circuit 16 selects one of the signal lines SGL in each of the signal line blocks at a time. Such a configuration can reduce the number of integrated circuits (ICs) including the detection circuit 48 or the number of terminals of the ICs in the detection device 1K. The signal line selection circuit 16 may couple more than one of the signal lines SGL collectively to the detection circuit 48.

As illustrated in FIG. 17, the reset circuit 17 includes a reference signal line Lvr, a reset signal line Lrst, and reset transistors TrR. The reset transistors TrR are provided correspondingly to the signal lines SGL. The reference signal line Lvr is coupled to either the sources or the drains of the reset transistors TrR. The reset signal line Lrst is coupled to the gates of the reset transistors TrR.

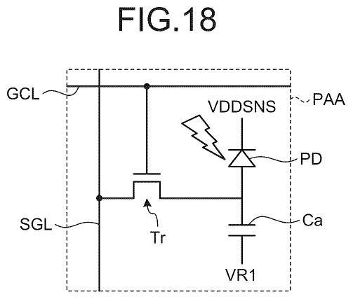

The control circuit 122 supplies a reset signal RST2 to the reset signal line Lrst. This operation turns on the reset transistors TrR to electrically couple the signal lines SGL to the reference signal line Lvr. The power supply circuit 123 supplies a reference signal COM to the reference signal line Lvr. This operation supplies the reference signal COM to a capacitive element Ca (refer to FIG. 18) included in each of the partial detection areas PAA.

FIG. 18 is a circuit diagram illustrating the partial detection area of the detection device according to the fifth embodiment. As illustrated in FIG. 18, the partial detection area PAA includes the photodiode PD, the capacitive element Ca, and a corresponding one of the drive transistors Tr. The capacitive element Ca is capacitance (sensor capacitance) generated in the photodiode PD and is equivalently coupled to the anode of the photodiode PD.

The drive transistor Tr is provided correspondingly to each of the photodiodes PD. The drive transistor Tr is configured as a thin-film transistor, and in this example, configured as an n-channel metal oxide semiconductor (MOS) thin-film transistor (TFT).

The gate of the drive transistor Tr is coupled to the gate line GCL. The source of the drive transistor Tr is coupled to the signal line SGL. The drain of the drive transistor Tr is coupled to the anode of the photodiode PD and the capacitive element Ca.

The cathode of the photodiode PD is supplied with the sensor power supply signal VDDSNS from the power supply circuit 123. The signal line SGL and the capacitive element Ca are supplied with a reference signal VR1 serving as an initial potential of the signal line SGL and the capacitive element Ca from the power supply circuit 123.

When the partial detection area PAA is irradiated with light in an exposure period, a current corresponding to the amount of the light flows through the photodiode PD. An electric charge to be stored in the capacitive element Ca depends on the current. After the drive transistor Tr is turned on in a read period, a current corresponding to the electric charge stored in the capacitive element Ca flows through the signal line SGL. The signal line SGL is coupled to the detection circuit 48 through the output transistor TrS of the signal line selection circuit 16. Thus, the detection device 1K can detect a signal corresponding to the amount of the light received by the photodiode PD in each of the partial detection areas PAA.

FIG. 19 is a plan view illustrating an arrangement relation between the photodiodes and the light sources in the detection device according to the fifth embodiment. As illustrated in FIG. 19, in the detection device 1K, the light-blocking wall 55 is provided in the peripheral area GA of the substrate 21 so as to surround the photodiodes PD, the gate line drive circuit 15, and the signal line selection circuit 16 in plan view. The light sources 53 and 54 are provided in the peripheral area GA of the substrate 21 and are provided between the light-blocking wall 55 and the outer edge of the substrate 21. The light sources 53 and 54 are arranged along each side of the peripheral area GA. For example, the light sources 53 and 54 are alternately arranged along one side of the peripheral area GA as follows: light source 53, light source 54, light source 53, light source 54. The light-blocking wall 56 is provided so as to surround the light sources 53 and 54.

In the following description, four photodiodes PD11, PD12, PD13, and PD14 among the photodiodes PD are illustrated and described to facilitate understanding. When the detection area AA is divided into four by an imaginary line 98 parallel to the first direction Dx and an imaginary line 99 parallel to the second direction Dy that pass through a central portion of the detection area AA, these four photodiodes PD correspond to a lower right area. The photodiodes PD are line-symmetrically arranged with respect to the imaginary line 98 parallel to the first direction Dx and the imaginary line 99 parallel to the second direction Dy that pass through the central portion of the detection area AA, and the description of the lower right area of the detection area AA can also be applied to the other areas.

The photodiode PD11 (first photodiode) located in the central portion of the detection area AA has a longer distance from the light sources 53 and 54. The photodiode PD12 (second photodiode) and the photodiode PD13 located in outer edge portions of the detection area AA have shorter distances from the light sources 53 and 54. The photodiode PD14 located at a corner among the photodiodes PD arranged in the outer edge portions of the detection area AA has shorter distances from two light sources 53 and 54, in more detail, from the light source 54 that is adjacent to the photodiode PD14 in the first direction Dx and the light source 53 that is adjacent to the photodiode PD14 in the second direction Dy.

In also the present embodiment, the light-receiving area of the photodiode PD increases as the distance from the light sources 53 and 54 increases, and the light-receiving area of the photodiode PD decreases as the distance from the light sources 53 and 54 decreases. Specifically, when focusing on the four photodiodes PD11, PD12, PD13, and PD14 described above, the light-receiving area of the photodiode PD11 (first photodiode) is larger than that of each of the photodiodes PD12 (second photodiode) and the photodiode PD13 (third photodiode). The photodiodes PD12 and the photodiode PD13 each have a shorter distance from the light sources 53 and 54 than that of the photodiode PD11. In addition, the light-receiving area of each of the photodiodes PD12 and PD13 is larger than that of the photodiode PD14 (fourth photodiode). The photodiode PD14 has a shorter distance from the light sources 53 and 54 than those of the photodiodes PD12 and PD13.

The area of a lower electrode 23-11 (sensor electrode) of the photodiode PD11 (first photodiode) located in the central portion of the detection area AA is larger than that of each of a lower electrode 23-12 (sensor electrode) of the photodiode PD12 (second photodiode) and a lower electrode 23-13 of the photodiode PD13 (third photodiode) that are located in the outer edge portions of the detection area AA. In addition, the area of each of the lower electrodes 23-12 and 23-13 of the photodiodes PD12 and PD13 is larger than that of the lower electrode 23-14 of the photodiode PD14 (fourth photodiode).

The area of each of the lower electrodes 23 gradually decreases from the photodiode PD11 located in the central portion of the detection area AA toward the photodiodes PD12, PD13, and PD14 located in the outer edge portions of detection area AA.

The area of each of the lower electrodes 23 arranged in the first direction Dx gradually decreases from the photodiode PD11 located in the central portion of the detection area AA toward the photodiode PD12. Similarly, the area of each of the lower electrodes 23 arranged in the second direction Dy gradually decreases from the photodiode PD11 located in the central portion of the detection area AA toward the photodiode PD13. Similarly, the area of each of the lower electrodes 23 arranged in a diagonal direction gradually decreases from the photodiode PD11 located in the central portion of the detection area AA toward the photodiode PD14.

The arrangement pitches Px of the lower electrodes 23 in the first direction Dx are equal from one end to the other end in the first direction Dx. The arrangement pitches Py of the lower electrodes 23 in the second direction Dy are equal from one end to the other end in the second direction Dy.

With such a configuration, even in the configuration in which the photodiodes PD are arranged in a matrix having a row-column configuration, the variations in detection sensitivity of the photodiodes PD due to the distance from the light sources 53 and 54 can be reduced. In other words, even the photodiode PD11 located in the central portion of the detection area AA and away from the light sources 53 and 54 can receive a sufficient amount of the light L1 for detecting the biometric information. Alternatively, the number of the light sources 53 and 54 required to emit a sufficient amount of the light L1 to the photodiode PD11 in the central portion of the detection area AA can be reduced.

In FIG. 19, both the width in the first direction Dx and the width in the second direction Dy of each of the lower electrodes 23 decreases from the central portion toward the outer edge portions of the detection area AA. However, the present disclosure is not limited to this configuration. The lower electrodes 23 may be provided so as to have different widths in only either one of the first direction Dx and the second direction Dy.

### Eighth Modification of Fifth Embodiment

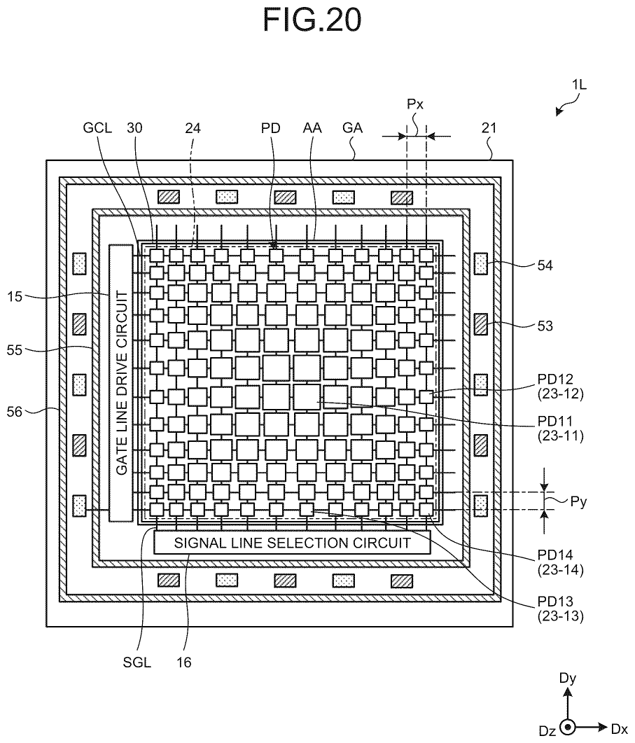

FIG. 20 is a plan view illustrating an arrangement relation between the photodiodes and the light sources in a detection device according to an eighth modification of the fifth embodiment. In the eighth modification of the fifth embodiment, the positional relation between the photodiodes PD and the light sources 53 and 54 is the same as that in the fifth embodiment described above (refer to FIG. 19). The configuration of a detection device 1L of the eighth modification differs from that in the fifth embodiment in that the arrangement pitches Px and Py of the photodiodes are set so as to vary from the central portion toward the outer edge portions of the detection area AA.

The arrangement pitch Px in the first direction Dx and the arrangement pitch Py in the second direction Dy of the lower electrodes 23 decrease as the distance from the light sources 53 and 54 decreases. Specifically, the arrangement pitch Px of the lower electrodes 23 arranged in the first direction Dx gradually decreases from the photodiode PD11 located in the central portion of the detection area AA toward the photodiode PD12. Similarly, the arrangement pitch Py of the lower electrodes 23 arranged in the second direction Dy gradually decreases from the photodiode PD11 located in the central portion of the detection area AA toward the photodiode PD13.

In the eighth modification, in the same way as in the fifth embodiment described above, the area of each of the lower electrode 23 gradually decreases from the photodiode PD11 located in the central portion of the detection area AA toward the photodiodes PD12, PD13, and PD14 located in the outer edge portions of the detection area AA. In FIG. 20, the light-receiving area of the photodiode PD14 located at a corner is set to be equal to the light-receiving area of each of the photodiode PD12 located in a central portion of one side and the photodiode PD13 located in a central portion of another side. However, the present disclosure is not limited to this configuration, and in the same way as in the example illustrated in FIG. 19, the light-receiving area (area of the electrode 23) may be set to be smaller as the distance from the central portion of the side increases from the photodiodes PD12 and PD13 located in the central portion of the one side and the central portion the other side toward the photodiode PD14 located at the corner.

With such a configuration, the light-receiving areas of the photodiodes PD, that is, the areas of the lower electrodes 23 in the eighth modification are larger than those in the fifth embodiment. In other words, the light-receiving area of the photodiode PD increases as the distance from the light sources 53 and 54 increases, and the light-receiving area of the photodiode PD decreases as the distance from the light sources 53 and 54 decreases. In addition, the area ratio of the lower electrodes 23 in the detection area AA can be increased as compared with the fifth embodiment. Therefore, as compared with the fifth embodiment described above, the amount of the light L1 incident on each of the photodiodes PD can be increased, and the detection sensitivity can be improved.

### Ninth Modification of Fifth Embodiment

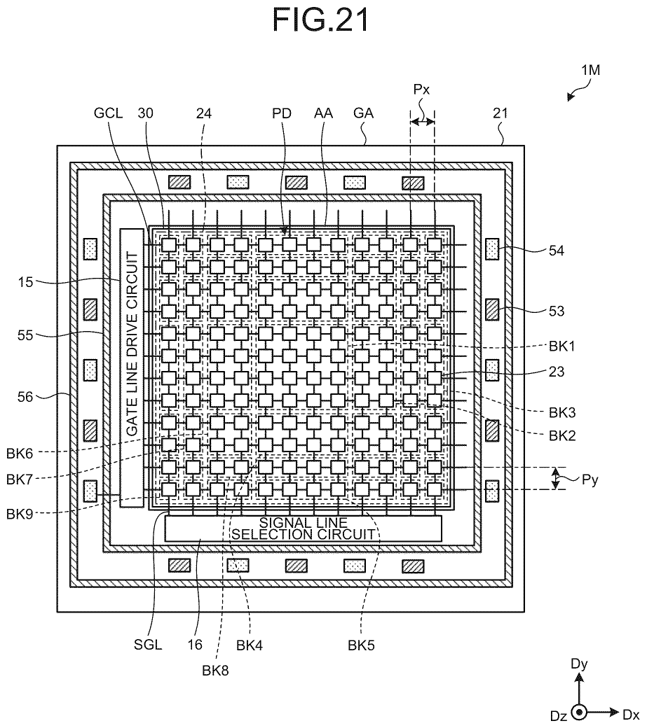

FIG. 21 is a plan view illustrating an arrangement relation between the photodiodes and the light sources in a detection device according to a ninth modification of the fifth embodiment. In the ninth modification of the fifth embodiment, the positional relation between the photodiodes PD and the light sources 53 and 54 is the same as that in the fifth embodiment described above (refer to FIG. 19). In the ninth modification, unlike the fifth embodiment and the eighth modification, the areas of the lower electrodes 23 of the photodiodes PD are set to be equal in the detection area AA, and the arrangement pitches Px and Py are set to be equal in the detection area AA.

In a detection device 1M of the ninth modification, the gate line drive circuit 15 collectively drives the gate lines GCL based on a control signal from the control circuit 122. The gate line drive circuit 15 drives the gate lines GCL such that the number of the gate lines GCL that are collectively driven is different between a central portion and outer edge portions in the second direction Dy of the detection area AA.

The signal line selection circuit 16 collectively couples the signal lines SGL to the detection circuit 48 (refer to FIG. 15) based on the selection signal ASW from the control circuit 122. The signal line selection circuit 16 couples the signal lines SGL to the detection circuit 48 such that the number of the signal lines SGL that are collectively coupled to the detection circuit 48 is different between a central portion and outer edge portions in the first direction Dx of the detection area AA.

Through the operations of the gate line drive circuit 15 and the signal line selection circuit 16, the signal line selection circuit 16 couples one of the photodiodes PD or collectively couples a plurality of the photodiodes PD as a sensor block BK to the detection circuit 48. The number of the photodiodes PD included in the sensor block BK1 located in the central portion of the detection area AA is larger than the number of the photodiodes PD included in a sensor block (such as a sensor block BK9) located in an outer edge portion of the detection area AA.

In more detail, the sensor block BK1 located in the central portion of the detection area AA includes 16 photodiodes PD, for example. Sensor blocks BK2 and BK3 that are closer to the outer edge in the first direction Dx of the detection area AA, that is, that each have a shorter distance from the light sources 53 and 54 than the sensor block BK1 include eight and four photodiodes PD, respectively.

Sensor blocks BK4 and BK5 that are closer to the outer edge in the second direction Dy of the detection area AA, that is, that each have a shorter distance from the light sources 53 and 54 than the sensor block BK1 include eight photodiodes PD and four photodiodes PD, respectively.

Sensor blocks BK6, BK7, BK8, and BK9 that are closer to the outer edges in the diagonal direction of the detection area AA, that is, that each have a shorter distance from the light sources 53 and 54 than the sensor block BK1 include four photodiodes PD, two photodiodes PD, two photodiodes PD, and one photodiode PD, respectively.

The signal line selection circuit 16 bundles together one or more of the photodiodes PD included in the sensor block BK and couples the bundle as a single sensor element to the detection circuit 48. As a result, in the detection device 1M, the light-receiving area of the sensor block BK1 located in the central portion of the detection area AA is formed to be larger, and the light-receiving area of a sensor block (such as the sensor block BK9) located in the outer edge portion of the detection area AA is formed to be smaller. In other words, the light-receiving area of the sensor block BK increases as the distance from the light sources 53 and 54 increases, and the light-receiving area of the sensor block BK decreases as the distance from the light sources 53 and 54 decreases.

The sensor blocks BK illustrated in FIG. 21 are merely exemplary, and the number of one or more of the photodiodes PD included in the sensor block BK can be changed as appropriate. In the fifth embodiment and each of the modifications thereof, the light-receiving areas of the photodiodes PD (sensor blocks BK) are symmetrically provided. This is, however, because the light sources 53 and 54 are evenly arranged around the detection area AA, and the light-receiving areas of the photodiodes PD (sensor block BK) can be changed depending on the arrangement of the light sources 53 and 54.

The shapes and areas of the lower electrodes 23 are equal, and the arrangement pitches Px and Py of the lower electrodes 23 are equal. The present disclosure is not limited to this configuration. The shapes, the areas, and the arrangement pitches Px and Py of the lower electrodes 23 may vary depending on the distance from the light sources 53 and 54. That is, the detection device 1M of the ninth modification can be combined with the configuration of the fifth embodiment or the eighth modification described above.

Each of the embodiments and the modifications thereof described above is merely exemplary and can be changed as appropriate. For example, in the first to the fourth embodiments, the examples have been illustrated in which the width Wx in the first direction Dx of the lower electrodes 23 is varied depending on the distance from the light sources 53 and 54, but the present disclosure is not limited to these examples. In the first to the fourth embodiments, the lower electrodes 23 may have the constant width Wx in the first direction Dx and different widths in the second direction Dy. Alternatively, in the first to the fourth embodiments, the lower electrodes 23 may have the different widths Wx in the first direction Dx and different widths in the second direction Dy.

The lower electrodes 23 all have a quadrilateral outer shape, but the outer shape is not limited thereto. The lower electrode 23 can have other shapes, such as a polygonal shape and a circular shape.

In each of the embodiments and the modifications thereof described above, the light-receiving areas of the photodiodes PD are symmetrically provided, but the present disclosure is not limited to this configuration.

In the first to the third embodiments and the modifications thereof described above, the lower electrode 23 is the anode electrode of the photodiode PD, and the upper electrode 24 is the cathode electrode of the photodiode PD. However, the present disclosure is not limited to this configuration. The lower electrode 23 may be the cathode electrode of the photodiode PD, and the upper electrode 24 may be the anode electrode of the photodiode PD. In that case, in the photodiode PD, the lower buffer layer 32 is configured with an electron transport layer, and the upper buffer layer 33 is configured with a hole transport layer.

While the preferred embodiments of the present disclosure have been described above, the present disclosure is not limited to the embodiments described above. The content disclosed in the embodiments is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiments and the modifications thereof described above.

What is claimed is:

1. A detection device comprising:
a light source configured to emit light to an object to be detected;
a plurality of photodiodes that each comprise a sensor electrode and an organic semiconductor layer and are arranged in a detection area; and
one or a plurality of detection circuits coupled to the photodiodes, wherein
the photodiodes comprise a first photodiode and a second photodiode that has a shorter distance from the light source than that of the first photodiode, and
a light-receiving area of the first photodiode is larger than a light-receiving area of the second photodiode.

2. The detection device according to claim 1, wherein an area of the sensor electrode of the first photodiode is larger than an area of the sensor electrode of the second photodiode.

3. The detection device according to claim 1, wherein an overlapping area where the sensor electrode of the first photodiode and the organic semiconductor layer overlap each other is larger than an overlapping area where the sensor electrode of the second photodiode and the organic semiconductor layer overlap each other.

4. The detection device according to claim 1, comprising a plurality of the light sources arranged around the detection area, wherein
an area of the sensor electrode of the first photodiode located in a central portion of the detection area is larger than an area of the sensor electrode of the second photodiode located in an outer edge portion of the detection area.

5. The detection device according to claim 1, comprising:
a plurality of signal lines coupled to the photodiodes; and
a signal line selection circuit configured to change coupling of the signal lines to the one or plurality of detection circuits, wherein
the signal line selection circuit is configured to collectively couple one or more of the photodiodes as a sensor block to the one or plurality of detection circuits, and
the number of the photodiodes included in the sensor block located in a central portion of the detection area is larger than the number of the photodiodes included in the sensor block located in an outer edge portion of the detection area.

6. The detection device according to claim 1, wherein
the photodiodes are arranged in a first direction, and
the light source is disposed outside the detection area so as to be adjacent in the first direction to the photodiodes.

7. The detection device according to claim 1, wherein
the photodiodes are arranged in a first direction, and
the light source is disposed outside the detection area so as to be adjacent to a central portion of the photodiodes arranged in the first direction.

8. The detection device according to claim 1, wherein the light source is configured to emit at least one of red light, infrared light, and green light.

9. The detection device according to claim 1, wherein the light source and the photodiodes are provided in an annular housing.

10. The detection device according to claim 9, wherein the light source and the photodiodes are provided on opposite sides with a center of the annular housing interposed between the light source and the photodiodes.

11. The detection device according to claim 9, wherein
the photodiodes are provided along a peripheral direction of the annular housing, and
an area of the sensor electrode of the first photodiode located in a central portion of the detection area is larger than an area of the sensor electrode of the second photodiode located in an outer edge portion in a peripheral direction of the detection area.

12. The detection device according to claim 9, wherein
the photodiodes are provided along a peripheral direction of the annular housing, and
an overlapping area where the sensor electrode of the first photodiode located in a central portion of the detection area and the organic semiconductor layer overlap each other is larger than an overlapping area where the sensor electrode of the second photodiode located in an outer edge portion in a peripheral direction of the detection area and the organic semiconductor layer overlap each other.
